SEQUENCE LISTING (16 sequences)

SEQ ID NO: 1 - protein, 7 aa, synthetic construct
GRGDSPC

SEQ ID NO: 2 - protein, 7 aa, synthetic construct
GRDGSPC

SEQ ID NO: 3 - protein, 16 aa, synthetic construct
GCRDVPMSMRGGDRCG

SEQ ID NO: 4 - protein, 4 aa, synthetic construct
AGAV

SEQ ID NO: 5 - protein, 5 aa, synthetic construct
PHSRN

SEQ ID NO: 6 - protein, 6 aa, synthetic construct
GFOGER

SEQ ID NO: 7 - protein, 15 aa, synthetic construct
GTPGPQGIAGQRGVV

SEQ ID NO: 8 - protein, 5 aa, synthetic construct
YIGSR

SEQ ID NO: 9 - protein, 5 aa, synthetic construct
IKVAV

SEQ ID NO: 10 - protein, 12 aa, synthetic construct
RKRLQVQLSIRT

SEQ ID NO: 11 - protein, 12 aa, synthetic construct
PPFLMLLKGSTR

SEQ ID NO: 12 - protein, 13 aa, synthetic construct
GKKQRFRHRNRKG

SEQ ID NO: 13 - protein, 7 aa, synthetic construct
FHRRIKA

SEQ ID NO: 14 - protein, 10 aa, synthetic construct
GWQPPARARI

SEQ ID NO: 15 - protein, 16 aa, synthetic construct
GCRDGPQGIWGQDRCG

SEQ ID NO: 16 - protein, 16 aa, synthetic construct
GCRDIPESLRAGDRCG

(19) United States
(12) Patent Application Publication
(10) Pub. No.: US 2024/0376438 A1
(43) Pub. Date: Nov. 14, 2024

(54) HYDROGELS COMPRISING CELL ADHESIVE PEPTIDES AND METHODS OF USE THEREOF

(71) Applicant: GEORGIA TECH RESEARCH CORPORATION, Atlanta, GA (US)

(72) Inventors: Susan Napier THOMAS, Atlanta, GA (US); Edward BOTCHWEY, Atlanta, GA (US); Andres J. GARCIA, Atlanta, GA (US); Meghan O'MELIA, Boston, MA (US)

(21) Appl. No.: 18/686,552

(22) PCT Filed: Aug. 26, 2022
(86) PCT No.: PCT/US2022/075515

§ 371 Date: Feb. 26, 2024

Related U.S. Application Data
(60) Provisional application No. 63/237,284, filed on Aug. 26, 2021.

(51) Int. Cl. C12N 5/09; A61L 27/18; A61L 27/38; A61L 27/52

(52) U.S. Cl. CPC C12N 5/0693; A61L 27/18; A61L 27/3804; A61L 27/52; A61L 2400/18; C12N 2533/52; C12N 2533/90; C12N 2537/10

(57) ABSTRACT

The present disclosure relates to a hydrogel biomaterial comprising cell adhesive peptides to generate in vivo tumor models.

### CROSS-REFERENCE TO RELATED APPLICATIONS

This application claims the benefit of U.S. Provisional Application No. 63/237,284, filed on Aug. 26, 2021, which is expressly incorporated herein by reference in its entirety.

### STATEMENT REGARDING FEDERALLY SPONSORED RESEARCH

This invention was made with government support under Grant Nos. R01CA207619, U01CA214354, R01AR062920, R01AR062368, T32GM008433, and T32EB006343 awarded by the National Institutes of Health (NIH). The government has certain right in the invention.

### FIELD

The present disclosure relates to a hydrogel biomaterial comprising cell adhesive peptides to generate in vivo tumor models.

### BACKGROUND

Immunotherapy has emerged as one of the most powerful anti-cancer therapy classes but is stymied by the limits of existing preclinical models with respect to disease latency and reproducibility. In addition, the influence of differing immune microenvironments within tumors observed in clinical disease and associated with immunotherapeutic resistance cannot be tuned to facilitate drug testing workflows without changing model system or laborious genetic approaches.

To address this testing platform gap in the immune oncology drug development pipeline, engineered biomaterials were deployed to increase tumor formation rate, decrease disease latency, and diminish variability of immune infiltrates into tumors formed from murine mammary carcinoma cell lines implanted into syngeneic mice. Given the divergence of cancer immunotherapies within a cancer model, there is need to address the problems mentioned above by developing biomaterials capable of inducing tumor models in animals to screen for specific cancer treatments.

The compounds, compositions, and methods disclosed herein address these and other needs.

### SUMMARY

The present disclosure relates to synthetic extracellular matrix compositions comprising cell adhesive peptides to induce tumors in a mouse and methods for the manufacture and use thereof.

In one aspect, disclosed herein is a cellular matrix scaffold comprising a poly(ethylene) glycol (PEG)-based hydrogel conjugated to a cell adhesive peptide, wherein the PEG hydrogel further comprises a crosslinking peptide and a population of cancer cells. Also disclosed are the cellular matrix scaffolds of any preceding aspect, wherein the cell adhesive peptide is selected from the group consisting of a fibronectin-derived peptide, a collagen-derived peptide, a laminin-derived peptide, a glycosaminoglycan binding peptide, and any other combination thereof. In some aspects, disclosed herein are the cellular matrix scaffolds of any preceding aspect, wherein the fibronectin-derived peptide is either a RGD peptide or a RDG peptide. In some embodiments, the RGD peptide comprises SEQ ID NO: 1. In some embodiments, the RDG peptide comprises SEQ ID NO: 2.

In another aspect, disclosed herein are the cellular matrix scaffolds of any preceding aspect, wherein the crosslinking peptide is a bis-cysteine crosslinking peptide. In some embodiments, the bis-cysteine crosslinking peptide is a VPM peptide. In some embodiments, the crosslinking peptide comprises SEQ ID NO: 3.

In another aspect, disclosed herein are the cellular matrix scaffolds of any preceding aspect, wherein the PEG is a four-armed maleimide-terminated PEG (PEG-4MAL) macromer. In another aspect, disclosed herein are the cellular matrix scaffolds of any preceding aspect, wherein the PEG, the cell adhesive peptide, the crosslinking peptide, and the population of cancer cells are combined at a 2:1:1:1 volume ratio.

In another aspect, disclosed are the cellular matrix scaffolds of any preceding aspects, wherein the population of cancer cells comprises either a population of breast cancer cells or a population of melanoma cells.

In one aspect, disclosed herein is a method of generating or inducing a tumor growth in an animal, the method comprising administering the cellular matrix scaffold of any preceding aspect into the animal. In some embodiments, the animal is a mammal. In some embodiments, the mammal is a rodent. In some embodiments, the rodent is a mouse.

In one aspect, disclosed herein is a kit to make a cellular matrix scaffold for screening anti-cancer drugs, the kit comprising a) a PEG hydrogel; b) a cell adhesive peptide powder; c) a crosslinking peptide powder; and d) a reconstitution buffer. In some embodiments, the cell adhesive peptide powder is resuspended with the reconstitution buffer to a concentration of 5 mM. In some embodiments, the reconstitution buffer is a 4-(2-hydroxyethyl)piperazine-1-ethanesulfonic acid (HEPES) buffer.

In another aspect, disclosed herein is a kit of any preceding aspect, wherein the kit comprises an immunomodulating agent. In another aspect, disclosed herein is a kit of any preceding aspect, wherein the kit comprises an adhesive ligand.

In one aspect, disclosed herein is a method of screening a drug, the method comprising a) generating the cellular matrix scaffold of any preceding aspect; b) administering the cellular matrix scaffold into an animal to induce a tumor; c) monitoring the animal for growth of the tumor; and d) administering to the animal a drug to treat the tumor. In another aspect, disclosed herein is a method of screening a drug of any preceding aspect, wherein the RGD peptide or the RDG peptide causes an immune cell to infiltrate the tumor. In some embodiments, the RGD peptide causes a T cell to infiltrate the tumor. In some embodiments, the T cell is a CD8+ T cell. In some embodiments, the RGD peptide causes a dendritic cell to infiltrate the tumor. In some embodiments, the dendritic cell is a CD86+ dendritic cell. In some embodiments, the RDG peptide causes a neutrophil cell to infiltrate the tumor. In some embodiments, the neutrophil cell is a CD11b+Ly6C+Ly6G+ neutrophil cell.

In another aspect, disclosed herein is a method of screening a drug of any preceding aspect, wherein the tumor is a breast tumor or another tumor type. In another aspect, disclosed herein is a method of screening a drug of any preceding aspect, wherein the method further comprises an immunomodulating agent. In another aspect, disclosed herein is a method of screening a drug of any preceding aspect, wherein the method further comprises an adhesive ligand. In another aspect, disclosed herein is a method of screening a drug of any preceding aspect, wherein the animal is a mammal. In some embodiments, the mammal is a rodent. In some embodiments, the rodent is a mouse.

### BRIEF DESCRIPTION OF DRAWINGS

The accompanying figures, which are incorporated in and constitute a part of this specification, illustrate several aspects described below.

FIGS. 1A-1K show the current preclinical models of breast cancer are insufficient. (FIG. 1a) Number of total cells in excised MMTV-PyMT tumors at week 8, 12, 16, and 20, and wild-type animals (week 0). a, each point represents one mouse. Rate of tumor formation (FIG. 1b) and tumor volume (FIG. 1c) after implantation of varying numbers of cells derived from tumors of MMTV-PyMT mice (backcrossed onto C57/Bl6 background) into mammary fatpad (MFP) of C57/Bl6 mice. FIG. 1b, each point represents one experiment with n=5 mice. FIG. 1c, each line represents the growth curve of one tumor implanted in one MFP of a mouse, n=5 per group. Rate of tumor formation (FIG. 1d) at two total Py230 cell doses, and tumor growth curves (e) of 250,000 Py230 cells implanted in the MFP of C57B16 mice in either saline or Matrigel (MT). FIG. 1d, each point represents one experiment with n=4-10 animals. FIG. 1e, data represents mean±s.e.m. with n=5 animals implanted with one tumor. Rate of tumor formation (FIG. 1f) at two total E0771 cell doses, and tumor growth curves (FIG. 1g) and time until tumors reach 100 mm³ (FIG. 1h) of 50,000 E0771 cells implanted in the MFP of C57/Bl6 mice in either saline and MT. f, each point represents one experiment with n=4 animals. FIG. 1g, data represents mean±s.e.m. with n=4 animals implanted with one tumor. h, each data point represents one tumor implanted animal. (FIG. 1i) Number of total lymphocytes (CD45+), macrophages (CD11b+F4/80+), and dendritic cells (CD11c+) at various times post implantation of 50,000 E0771 tumor cells in saline or MT into the MFP of C57B16 mice. Each data point represents one tumor implanted animal. Number of total lymphocytes (CD45+), macrophages, dendritic cells, and T cells (FIG. 1j) and phenotype of macrophages, DCs, and T cells (k) at day 7 after 50,000 E0771 cells were implanted in the MFP of C57/Bl6 mice in saline or two different batches of MT. Each data point represents one tumor implanted animal. * indicates significance by two-way ANOVA (FIG. 1b) or mixed-effects analysis (FIG. 1d, FIG. 1f, FIG. 1i, FIG. 1j, FIG. 1k) with Tukey's post-hoc comparison; # indicates significance by RM ANOVA with Tukey's post-hoc comparison; \hat{ } indicates significant difference in variance by Brown-Forsythe's test. * \hat{ } indicate p<0.05, **, \hat{ }\hat{ } indicate p<0.01, *** indicates p<0.005, **** indicates p<0.001.

FIGS. 2A-2D show the survival of animals after implantation of 250,000 Py230 cells (FIG. 2a) and 50,000 E0771 cells (FIG. 2b) in different batches of MT into the MFP of C57/Bl6 mice. n=4 animals per group. (FIG. 2c) Growth curves of 50,000 E0771 cells implanted in saline and MT into the MFP of C57B16 mice. Data represents mean±s.e.m. of n=4 animals implanted with one tumor. (FIG. 2d) Growth curves of 50,000 E0771 cells implanted into the MFP of C57B16 mice in one of two different batches of PEG hydrogel functionalized with either RGD or RDG. Data represent data represents mean±s.e.m. of n=4 animals implanted with one tumor. # indicates significance by RM ANOVA with Tukey's post-hoc test; #### indicates p<0.001.

FIG. 3 shows the gating strategy for T cell panel, shown in representative lymph node sample.

FIG. 4 shows the gating strategy for antigen-presenting/myeloid cell panel, shown in representative lymph node sample.

FIGS. 5A-5F show engineered hydrogels induce consistent and controllable tumor immune microenvironments. (FIG. 5a) Schematic diagram of hydrogels consisting of PEG-4MAL, with VPM crosslinkers, and adhesive ligands (stars). Tumor formation (FIG. 5b) and growth (FIG. 5c) rate after implantation of 50,000 E0771 cells into C57B16 mice in PEG hydrogel matrix vehicle. FIG. 5b, each point represents one experiment with n=4 mice. c, measured as time to 100 mm³ in tumor volume. Each data point represents one tumor implanted animal. (FIG. 5d) Number of total lymphocytes (CD45+), macrophages (CD11b+F4/80+), and DCs (CD11c+) within tumor various times post implantation of 50,000 E0771 cells into C57B16 mice in PEG hydrogel matrix vehicle. Each point represents one animal. (FIG. 5e) Degradation of AF750-labelled PEG matrix vehicle implanted into C57B16 mice relative to d0 signal, measured by IVIS imaging, with or without co-implantation of 50,000 E0771 cells. Data represents mean±s.e.m. with n=6 animals implanted with one tumor. (FIG. 5f) Number of total lymphocytes (CD45+), macrophages, dendritic cells, and T cells within tumor day 7 post implantation of 50,000 E0771 cells into C57B16 mice in two separate PEG hydrogel matrix vehicle batches prepared ˜1 year apart. Each data point represents one tumor implanted animal. For all groups, error bars indicate mean±s.e.m. * indicates significance by one-way ANOVA with Tukey's post-hoc comparison; * indicates p<0.05, n.s. indicates not significant (p>0.05).

FIGS. 6A-6I show the adhesive ligands alter immune responses in tumors formed using engineered hydrogel matrix vehicles. (FIG. 6a) VEGF-A concentration within tumors formed from 50,000 E0771 cells implanted in C57B16 mice in PEG hydrogel matrix vehicles at d2, 7, and 28 after implantation. Each data point represents one tumor implanted animal. Vasculature within tumors formed from 50,000 E0771 cells implanted in C57B16 mice in PEG hydrogel matrix vehicles, as measured by micro-computed tomography, visualized in (FIG. 6b) and quantified in (FIG. 6c). FIG. 6b, scale bar indicates 100 m. FIG. 6c, each data point represents one tumor implanted animal. Tumor growth curves (FIG. 6d), days until a tumor volume of 100 mm³ was reached (FIG. 6e) and animal survival (FIG. 6f) after 50,000 E0771 cells were implanted in C57/Bl6 mice in PEG hydrogel matrix vehicles. FIG. 6d, data represent mean±s.e.m. of n=4 tumors implanted into individual animals. FIG. 6e, each data point represents one tumor implanted animal. FIG. 6f, n=4. (FIG. 6g) Histological sections of tumors 2 and 7 days after implantation of 50,000 E0771 cells into C57/Bl6 mice in either PEG hydrogel matrix vehicle formulation. Images are representative of n=4 animals per group, scale bars represent 100 μm. Tumor growth curves (FIG. 6h) and days until a tumor volume of 100 mm³ was reached (FIG. 6i) after 50,000 E0771 cells were implanted into NSG mice in various matrix vehicle types. FIG. 6h, data represent mean±s.e.m. of n=4 tumors implanted into individual animals. FIG. 6i, each data point represents one tumor implanted animal. * indicates significance by one-way ANOVA (FIG. 6i) or mixed-effects analysis (FIG. 6a, FIG. 6c) with Tukey's post-hoc comparison, or Mann-Whitney test (FIG. 6e); # indicates significance by RM ANOVA with Tukey's post-hoc test; $ indicates significance by Log-Rank test; * indicates p<0.05, ** indicates p<0.01, $$$ indicates p<0.005, #### indicates p<0.001, n.s. indicates not significant (p>0.05).

FIGS. 7A-7H show the hydrogel adhesive ligands alter immune responses in tumors formed within synthetic hydrogel matrix vehicles. CD206/CD86 ratio among DCs (FIG. 7a), M2/M1 ratio among macrophages (FIG. 7b), number of CD11b+Ly6C+Ly6G+ cells (FIG. 7c), CD8/Treg ratio among T cells (FIG. 7d), and number of CD44-CD8+ T cells (FIG. 7e) infiltrating tumors at d2, 7, and 28 after implantation of 50,000 E0771 cells into C56B16 mice in varying PEG hydrogel matrix vehicle formulations. Each data point represents one tumor implanted animal. (FIG. 7f) Resulting immune infiltration from RGD (top) and RDG (bottom) ligands. (FIG. 7g-FIG. 7h) Tumor cytokine levels at d2, 7, and 28 after implantation of 50,000 E0771 cells into C57/Bl6 mice within PEG hydrogel matrix vehicles. g, each column represents an individual animal, and levels are scaled from minimum (blue) to maximum (red) in each row. FIG. 7h, each data point represents one tumor implanted animal. * indicates significance by one-way ANOVA (FIG. 7a, FIG. 7b, FIG. 7e) or mixed-effects analysis (FIG. 7c, FIG. 7d, FIG. 7h) with Tukey's post-hoc comparison; statistics for g in Table 1; * indicates p<0.05, ** indicates p<0.01, *** indicates p<0.005, ND indicates no data (not enough cells counted for reliable result).

FIGS. 8A-8F show the immune remodeling in lymphoid tissues with scaffold implantation. CD206+/CD86+ ratio of CD11c+ cells in draining lymph node [dLN] (FIG. 8a) and spleen (FIG. 8b); ratio of CD8+ T cells to CD4+ Tregs in dLN (FIG. 8c) and spleen (FIG. 8d); and number of CD11b+Ly6C+Ly6G+ cells in dLN (FIG. 8e) and spleen (FIG. 8f) after implantation of 50,000 E0771 cells in the MIFP of C57/Bl6 mice in the indicated matrix vehicle. Each data point represents one tumor implanted animal. * indicates significance by two-way ANOVA with Tukey's post-hoc comparison; n=4 mice. * indicates p<0.05, ** indicates p<0.01, *** indicates p<0.005, **** indicates p<0.001.

FIGS. 9A-9C show the immune status is affected by both tumor- and adhesive ligand-specific responses. CD206+/CD86+ ratio of CD11c+ cells (FIG. 9a), CD8+ T cell: Treg ratio (FIG. 9b), and FIG. 9 (c) number of CD11b+Ly6C+Ly6G+ cells in PEG-RGD and PEG-RDG scaffolds in C57/Bl6 mice with or without co-implantation of 50,000 E0771 cells. Each data point represents one scaffold implanted animal. * indicates significance by mixed-effects analysis with Tukey's post-hoc comparison; n=4 mice; * indicates p<0.05, ** indicates p<0.01.

FIGS. 10A-10F show the tumor response to immunotherapy is dependent upon matrix vehicle. Tumor growth curves (FIG. 10a, FIG. 10c, FIG. 10e) and animal survival (FIG. 10b, FIG. 10d, FIG. 10f) of C57/Bl6 mice implanted with E0771 cells after CpG vaccine administered i.t. on day 0 and 7 (FIG. 10a-FIG. 10b), combination αPD1+αCTLA4 mAb treatment administered i.p. on days 0, 3, and 6 (FIG. 10c-FIG. 10d), and isotype control mAb administered i.p. on days 0, 3, and 6 (FIG. 10e-FIG. 10f). a, c, e, data represent mean±s.e.m. of n=5-7 tumors implanted into individual animals. d0 signifies the first day of treatment when tumors reached 100 mm³. Arrows indicate days therapy is given. * indicates significance by repeated measures ANOVA with Tukey's post-hoc test, against all other groups if not specified; $ indicates significance against all other groups by log-rank test. FIG. 10a, FIG. 10b, FIG. 10e, and FIG. 10f are representative of two independent experiments; *, $ indicate p<0.05, ***, $$$ indicate p<0.005; n.s. indicates not significant (p>0.05).

FIGS. 11A-11C show the individual growth curves after CpG vaccine administered i.t. on day 0 and 7 (FIG. 11a), combination αPD1+αCTLA4 mAb treatment administered i.p. on days 0, 3, and 6 (FIG. 11b), and isotype control mAb administered i.p. on days 0, 3, and 6 (FIG. 11c). Tumors were formed from 50,000 E0771 cells implanted into C57/Bl6 mice within the indicated matrix vehicle, with d0 signifying the first day of treatment (when tumors reached 100 mm³). n=5-7 per group. Arrows indicate days therapy was given.

FIGS. 12A-12B show the growth of tumors formed after implantation of 50,000 E0771 cells into C57/Bl6 mice within PEG-RGD (FIG. 12a) and PEG-RDG (FIG. 12b) hydrogels matrix vehicles beginning at tumor implantation and treated with it. CpG, i.p. anti-PD1+anti-CTLA4, i.p. isotype mAb control, or untreated. Treatment groups are from experiment independent of FIG. 6; untreated group is from gel degradation study. Data represents mean±s.e.m. with n=5-7 animals implanted with one tumor. Each animal is treated on the day the tumor reaches 100 mm³ in volume (noted as a dashed line). * indicates significance by repeated measures ANOVA with Tukey's post-hoc test, against all other groups if not specified. * indicates p<0.05, *** indicates p<0.005.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, “polypeptide” or “peptide” refers to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like. Also as used herein, the term “adhesive peptide” refers to a peptide that promotes adhesion of normal and/or tumor cells and are derived from extracellular matrix glycoproteins such as laminin, fibronectin, and collagen.

As used herein, “therapeutic” generally can refer to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “kit” describes a wide variety of bags, containers, carrying cases, and other portable enclosures which may be used to carry and store solid substances, liquid substances, and other accessories necessary to induce and build a tumor in an animal. Such kits and their contents along with any applicable procedures may be used to provide access to animal models in accordance with the teachings of the present disclosure.

As used herein, the term “agent” refers to a living organism or biological substance, such as a bacterium, virus, protozoan, parasite, fungus, chemical, or toxin, that can design to purposefully fulfill a biological function or action.

The term “cancer” is used to address any neoplastic disease. It is used here to describe both solid tumors and hematologic malignancies, including for example, epithelial (surface and glandular) cancers, soft tissue and bone sarcomas, angiomas, mesothelioma, melanoma, lymphomas, leukemias and myeloma.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments may be applied preventively, prophylactically, pallatively or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

As used herein, “control” is an alternative subject or sample used in an experiment for comparison purposes and included to minimize or distinguish the effect of variables other than an independent variable. A “control” can be positive or negative.

As used herein, “active agent” or “active ingredient” refers to a component or components of a composition to which the whole or part of the effect of the composition can be attributed.

As used herein, “immune response” refers to the action of the immune system including immune cells and macromolecules produced by these cells that leads to selective targeting and destruction of pathogens or cancer cells and healthy cells in the case of autoimmunity.

As used herein, “biologic” refers to a large, complex molecule including antibody proteins, both whole and fragments.

As used herein, “immunotherapy” refers to a class of therapy meant to modulate or elicit immune responses in a curative or amelioratory capacity.

As used herein, “tumor immune micro-environment” refers to a tumor and localized immune cells, immunomodulatory molecules, and other interactions between the immune system and a tumor.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

A “matrix scaffold” refers to a complex mixture of structural and functional polymers that serves an important role in tissue and organ morphogenesis, maintenance of cell and tissue structure and function, and in the host response to injury. As used herein, the matrix scaffold refers to a mixture of polymers engineered to support tumor growth and maintenance to generate cancer models in an animal. The matrix scaffolds also provide the structural support for cell attachment and contribute to formation of local microenvironment. The polymers utilized in the matrix scaffold can include protein polymers, synthetic polymers, or any combination thereof.

The terms “crosslink”, “crosslinking” or “crosslinker(s)” refers to the process of chemically joining two or more molecules by a covalent bond. Crosslinking reagents (also termed crosslinkers) are molecules that contain two or more reactive ends capable of chemically attaching to specific functional groups (i.e.: primary amines, sulfhydryls, carboxyls, etc.) on proteins or other molecules. When a polymer is crosslinked, a bond is formed between polymer chains, either between different chains or between parts of the same chain.

As used herein, “vaccine” refers to aa substance or composition used to stimulate the production of antibodies and provide immunity against one or several diseases, prepared from the causative agent of a disease, its products, or a synthetic substitute, treated to act as an antigen without inducing the disease. A vaccine is also a preparation that is administered (usually by injection) to stimulate the body's immune response against a specific infectious agent or disease: such as an antigenic preparation of a typically inactivated or attenuated pathogenic agent (such as a bacterium or virus) or one of its components or products (such as a protein or toxin).

As used herein, the term “volume ratio” refers to a quantification of certain solute or group of solutes, measure by volume, in a solution. This term can also be defined as the sum of the volume of solute A, volume of solute B, volume of solute N, etc., wherein all solutes equal or total 100% of the total volume.

As used herein, a “hydrogel” refers to a three-dimensional polymer network to imbibe large amounts of water, which is used for the purpose of biomedical applications, including but not limited to: treatment of wound healing, cell culture, drug delivery, contact lenses, plastic surgery, and tissue regeneration. Hydrogels can chemically or physically contain various pharmaceutical drugs or immunomodulatory agents including chemical drugs, proteins, peptides, nucleotides, and ions. Hydrogels also enable control of responses by modulation of the density and polarity of the polymer network, imparting the stimuli responsiveness to the polymer network.

As used herein, “poly(ethylene) glycol” or “PEG” refers to a synthetic, hydrophilic, biocompatible polymer made by joining of ethylene oxide and water molecules. In part due to its biocompatibility, PEG can be used in tissue engineering applications.

As used herein, “T cell” refers to a lymphocyte produced by the thymus glad that resides in lymph nodes. T cells play a major role in cell-mediated immunity which is limited by their specificity toward antigens due to their T cell receptor and cytotoxic mechanisms to eliminate infected or mutated cells. T cells play a major role in cancer immunotherapy.

As used herein, “dendritic cell” refers to a type of cell produce by bone marrow which are specialized to capture and process antigen, acting as professional antigen-presenting cells, capable of activating and modulating the phenotype of native T cells.

As used herein, “macrophage” refers to a professional phagocytic cell specialized in removing cellular debris, capable of activating and modulating other lymphocytes either directly or via cytokine production.

As used herein, “monitoring” refers to the actions of observing and checking the progress or quality of a treatment or procedure over a period of time. “Monitoring” also refers to observing the course of a disease or condition, such as a cancer, over a period of time.

As used herein, the term “buffer” refers to a solution consisting of a mixture of acid and its conjugate base, or vice versa. The solution is used as a means of keeping the pH at a nearly constant range to be used in a wide variety of chemical and biological applications.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions and Scaffolds

The present disclosure relates to cellular matrix compositions comprising cell adhesive peptides to induce tumors in a mouse and methods for the manufacture and use thereof.

Hydrogels are commonly used in biomedical applications, such as drug delivery, tissue engineering, cell encapsulation matrices because their physical properties, whether synthetic or natural, are similar to natural tissues. Hydrogels are physically or chemically crosslinked hydrophilic polymers molecules that do not dissolve in water, but instead exhibit a high degree of swelling in an aqueous environment. Polyethylene glycol (PEG) is an example of a synthetic hydrogel polymer commonly used in tissue regeneration and targeted drug delivery systems. The PEG macromer and variations that exist thereof exhibit versatile properties with exceptional biocompatibility. When properly designed and created, PEG macromers are essential for directing cellular functions that are important for survival, proliferation, differentiation, and secretory properties. To construct PEG macromers, crosslinking molecules, such as peptides, are used to provide mechanical, thermal, and chemical stabilization to the hydrogel. Furthermore, addition of cell adhesive peptides to a PEG hydrogel supports tissue regeneration and engineering by binding adhesion receptors promoting cell survival, migration, proliferation, and expression of different phenotypes and secretory functions. In the present disclosure, a four-armed maleimide-terminated PEG macromer is conjugated with a cell adhesive peptide and a crosslinking peptide to generate a hydrogel composition intended to induce tumor growth in an animal model.

In one aspect, disclosed herein is a cellular matrix scaffold comprising a polyethylene glycol (PEG) hydrogel conjugated to a cell adhesive peptide, wherein the PEG hydrogel further comprises a crosslinking peptide and a population of cancer cells. Also disclosed are the cellular matrix scaffolds of any preceding aspect, wherein the cell adhesive peptide is selected from the group consisting of fibronectin-derived peptides, collagen-derived peptides, laminin-derived peptides, glycosaminoglycan binding peptides, and any other combination thereof. In some aspects, disclosed herein are the cellular matrix scaffolds of any preceding aspect, wherein the fibronectin-derived peptide is either a RGD peptide or a RDG peptide. In some embodiments, the RGD peptide comprises SEQ ID NO: 1. In some embodiments, the RDG peptide comprises SEQ ID NO: 2. In some embodiments, the fibronectin-derived peptide is a REDV peptide (SEQ ID NO: 4) or a PHSRN peptide (SEQ ID NO: 5). In some embodiments, the collagen-derived peptide is a GFOGER peptide (SEQ ID NO: 6) or a P-15 peptide (SEQ ID NO: 7). In some embodiments, the laminin-derived peptide is a YIGSR peptide (SEQ ID NO: 8), an A5G81 peptide, an IKVAV peptide (SEQ ID NO: 9), an AG73 peptide (SEQ ID NO: 10), or a P4 peptide (SEQ ID NO: 11). In some embodiments, the glycosaminoglycan binding peptide is a GKKQRFRHRNRKG (SEQ ID NO: 12), FHRRIKA (SEQ ID NO: 13), or GWQPPARARI (SEQ ID NO: 14).

In another aspect, disclosed herein are the cellular matrix scaffolds of any preceding aspect, wherein the crosslinking peptide is a bis-cysteine crosslinking peptide. In some embodiments, the bis-cysteine crosslinking peptide is a VPM peptide. In some embodiments, the crosslinking peptide comprises SEQ ID NO: 3. In further embodiments, the crosslinker can comprise GPQ-W (SEQ ID NO: 15). In some embodiments, the crosslinker can comprise IPES (SEQ ID NO: 16).

In another aspect, disclosed herein are the cellular matrix scaffolds of any preceding aspect, wherein the PEG is a four-armed maleimide-terminated PEG (PEG-4MAL) macromer. In another aspect, disclosed herein are the cellular matrix scaffolds of any preceding aspect, wherein the PEG, the cell adhesive peptide, the crosslinking peptide, and the population of cancer cells are combined at a 2:1:1:1 volume ratio.

In another aspect, disclosed are the cellular matrix scaffolds of any preceding aspects, wherein the population of cancer cells comprises a population of breast cancer cells. In another aspect, disclosed are the cellular matrix scaffolds of any preceding aspects, wherein the population of cancer cells comprises a population of melanoma cells. In some embodiments, the population of cancer cells comprises a population of lymphoma cancer cells, sarcoma cancer cells, carcinoma cancer cells, leukemia cancer cells, brain cancer cells, or spinal cord cancer cells.

In some aspects, disclosed herein is a polyethylene (PEG) hydrogel, wherein the hydrogel has been functionalized with RGD or RDG peptides and further wherein the hydrogel comprises cancer cells. In some embodiments, the cancer cells are breast cancer or melanoma cells. In some embodiments, the hydrogel further comprises a bis-cysteine crosslinking peptide. In some embodiments, the RGD peptide is GRGDSPC (SEQ ID NO: 1) and the RDG peptide is GRDGSPC (SEQ ID NO: 2). In some embodiments, the bis-cysteine crosslinking peptide is GCRDVPMSMRGGDRCG (SEQ ID NO: 3).

### Kits

A kit incorporating content and components of the present disclosure provides a convenient way to carry said content and components in an operable way to achieve tumor growth in an animal in an organized and systematic fashion. In many applications, a kit is designed for use in biomedical, biomechanical, tissue engineering, and any related applications.

In one aspect, disclosed herein is a kit to make a cellular matrix scaffold for screening anti-cancer drugs, the kit comprising a) a PEG hydrogel; b) a cell adhesive peptide powder; c) a crosslinking peptide powder; and d) a reconstitution buffer. In some embodiments, the cell adhesive peptide powder is resuspended with the reconstitution buffer to a concentration of 5 mM. In some embodiments, the crosslinking peptide powder is resuspended with the reconstitution buffer. In further embodiments, the bis-cysteine crosslinking peptide (SEQ ID NO: 3) can be used at a density corresponding to 1:1 maleimide to cysteine ratio after accounting for maleimide groups reacted with adhesive peptide. In some embodiments, the reconstitution buffer is a 4-(2-hydroxyethyl)piperazine-1-ethanesulfonic acid (HEPES) buffer. In further embodiments, the buffer is phosphate-buffered saline (PBS). In some embodiments, the buffer can include any buffer in the physiological range of 6.5-7.5.

An immune response is initiated in response to invasion of foreign pathogens in a host. In response to cancer cells, the adaptive immune system is activated comprising B lymphocytes and T lymphocytes. However, cancer cells develop properties to evade immune responses thereby requiring immunomodulating agents to further stimulate an anti-cancer immune response. In another aspect, disclosed herein is a kit of any preceding aspect, wherein the kit comprises an immunomodulating agent. In some embodiments, the immunomodulating agent is a chemokine or a cytokine. In some embodiments, the chemokine includes, but is not limited to CCL2, CCL1, CCL19, CCL22, CXCL12, CCL17, MIP-1α, MCP-1, GRO/KC, and/or CXCR3. In some embodiments, the cytokine includes, but is not limited to IL-1β, IL-2, IL-4, IL-6, IL-8, IL-10, IL-12, IL-15, IL-18, IL-21, IL-22, IFN-γ, TNF-α, TGF-β, LIF, cytotoxins, perform, and/or granzyme. Adhesive ligands can also be introduced to promote regression of tumor growth. These cell adhesive molecules have been shown to promote or antagonize tumor growth; however, selection of the appropriate adhesive molecules is required to prevent cancer growth. In another aspect, disclosed herein is a kit of any preceding aspect, wherein the kit comprises an adhesive ligand. In some embodiments, the adhesive ligand is an immunoglobulin-like adhesion molecule, an integrin, a cadherin, or a selectin.

### Methods

Current systems for cancer immunotherapeutic testing include patient-derived xenografts, in which a portion of a patient's tumor is excised and implanted into humanized or immunodeficient animals, but which can be prohibitively expensive and do not necessarily correlate with clinical responses; genetically engineered mouse models, in which animals are genetically modified to express tumor-inducing molecules under control of certain portions of the genome, but which can be highly variable and inconsistent; and syngeneic tumor models, in which tumor cells are injected into syngeneic, immunocompetent hosts in either Matrigel or saline, but which likewise can have variable and uncontrollable immune responses. This system utilized an engineered and consistent system allowing for consistent tumor formation and development, along with consistent and controllable immune infiltration.

In one aspect, disclosed herein is a method of generating or inducing a tumor growth in an animal, the method comprising administering the cellular matrix scaffold of any preceding aspect into the animal. In some embodiments, the animal is a bird, a fish, or a frog. In some embodiments, the animal is a mammal. In some embodiments, the mammal is a dog, a cat, a cow, a horse, a pig, a rabbit, a sheep, or a monkey. In some embodiments, the mammal is a rodent. In some embodiments, the rodent is a guinea pig, a hamster, or a rat. In some embodiments, the rodent is a mouse.

Cancers are known to form and utilize a variety of genetic and epigenetic mutations, leading to the formation of neoantigens, which can be recognized by the immune system. The adaptive immune system, composed of B and T lymphocytes has the potential to eliminate cancer due to the broad recognition of cancer neoantigens and effective cytotoxic functions. Utilization of these features is why immunotherapies have emerged as one of the most promising tools in the fight against cancer. However, response rates in the clinic are low; only ˜16% of patients respond to immune checkpoint blockade (ICB) therapy, and vaccine therapies have had limited success. This shows an unmet need in understanding the differences in patient responses, and tools to develop new and better immunotherapeutic strategies for patients who are less likely to respond to immunotherapies.

In one aspect, disclosed herein is a method of screening a drug, the method comprising a) generating the cellular matrix scaffold of any preceding aspect; b) administering the cellular matrix scaffold into an animal to induce a tumor; c) monitoring the animal for growth of the tumor; and d) administering to the animal a drug to treat the tumor. In another aspect, disclosed herein is a method of screening a drug of any preceding aspect, wherein the RGD peptide or the RDG peptide causes an immune cell to infiltrate the tumor. In some embodiments, the RGD peptide causes a T cell to infiltrate the tumor. In some embodiments, the T cell is a CD8+ T cell. In some embodiments, the T cell is a CD4+ T cell. In some embodiments, the RGD peptide causes a dendritic cell to infiltrate the tumor. In some embodiments, the dendritic cell is a CD86+ dendritic cell. In some embodiments, the RDG peptide causes a neutrophil cell to infiltrate the tumor. In some embodiments, the neutrophil cell is a CD11b+Ly6C+Ly6G+ neutrophil cell. In further embodiments, other markers can be used to define neutrophil cells.

In another aspect, disclosed herein is a method of screening a drug of any preceding aspect, wherein the tumor is a breast tumor or a melanoma tumor. In some embodiments, the tumor is a lymphoma, a sarcoma, a carcinoma, a leukemia, a brain tumor, or a spinal cord tumor. In another aspect, disclosed herein is a method of screening a drug of any preceding aspect, wherein the method further comprises administering an immunomodulating agent. In some embodiments, the immunomodulating agent is a chemokine or a cytokine. In some embodiments, the chemokine includes, but is not limited to CCL2, CCL1, CCL19, CCL22, CXCL12, CCL17, MIf-1α, MCP-1, GRO/KC, and/or CXCR3. In some embodiments, the cytokine includes, but is not limited to IL-1β, IL-2, IL-4, IL-6, IL-8, IL-10, IL-12, IL-15, IL-18, IL-21, IL-22, IFN-γ, TNF-α, TGF-β, LIF, cytotoxins, perform, and/or granzyme. In another aspect, disclosed herein is a method of screening a drug of any preceding aspect, wherein the method further comprises an adhesive ligand. In some embodiments, the adhesive ligand is an immunoglobulin-like adhesion molecule, an integrin, a cadherin, or a selectin. In another aspect, disclosed herein is a method of screening a drug of any preceding aspect, wherein the animal is a mammal. In some embodiments, the animal is a bird, a fish, or a frog. In some embodiments, the mammal is a dog, a cat, a cow, a horse, a pig, a rabbit, a sheep, or a monkey. In some embodiments, the mammal is a rodent. In some embodiments, the rodent is a guinea pig, a hamster, or a rat. In some embodiments, the rodent is a mouse.

In some aspects, disclosed herein is a method of generating tumors in an animal, the method comprising implanting in the animal a hydrogel, wherein the hydrogel is functionalized with RGD or RDG peptides and cancer cells. In some embodiments, the hydrogel is a PEG hydrogel. In some embodiments, the animal is a mouse. In some embodiments, the animal is a model for immunotherapeutic experimentation. In some embodiments, the cancer cells are breast cancer or melanoma cells. In some embodiments, the hydrogel further comprises a bis-cysteine crosslinking peptide. In some embodiments, the RGD peptide is GRGDSPC (SEQ ID NO: 1) and the RDG peptide is GRDGSPC (SEQ ID NO: 2). In some embodiments, the bis-cysteine crosslinking peptide is GCRDVPMSMRGGDRCG (SEQ ID NO: 3).

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Biomaterial-Programmed Tumor Immune Microenvironments for Preclinical In Vivo Cancer Immunotherapy Evaluation

Breast cancer is the most common cancer among women worldwide, affecting approximately 2.3 million people annually. Triple-negative breast cancer (TNBC), which lacks hormone receptor and excess HER2 protein expression, is the most aggressive type of breast cancer, with higher rates of metastasis and shorter overall survival. Immunotherapies have emerged as one of the most promising tools in the fight against breast cancer. However, response rates in the clinic are disappointingly low—only ˜16% of patients respond to immune checkpoint blockade (ICB) therapy⁴, and no vaccine therapies have been approved for breast cancer, despite successes in melanoma and prostate cancer. These poor outcomes reflect an unmet need in understanding the differences in patient responses and tools to develop new and better immunotherapeutic strategies for patients who are less likely to respond to immunotherapies.

Substantial efforts in the immune oncology field are currently dedicated to unraveling the determinants of the anti-cancer immune response, given its role in disease progression and response to immunotherapy. Immunologically “hot” tumors generally have more immune infiltration, while “cold” tumors harbor fewer immune cells. Additionally, other stratifications have been identified, with the potential to influence immunotherapy responses—among immune-infiltrated (hot) tumors, macrophages and CD8 T cells have been shown to have opposite impacts on patient survival, and in turn on responses to chemotherapy. Likewise, neutrophils and CD8 T cells form unique signatures that induce differing ICB responses, with CD8 T cell-infiltrated tumors demonstrating much higher responsiveness to ICB. Additionally, T cell programmed death-1 (PD1) expression impacts survival opposite to regulatory T cell infiltration, with implications for ICB responses. Thus, even within immunologically “hot” tumors, different immune cell signatures have potential ramifications on patient survival as well as responses to immunotherapy and chemotherapy. This variability in local immune microenvironment is thought to underlie immunotherapeutic resistance, including in the context of TNBC, motivating patient screening approaches to identify therapies with the highest curative potential.

Due to their reliance on the coordinated effects of multiple arms of the immune system, immunotherapies are tested primarily using preclinical animal models. Current breast cancer models include patient-derived xenografts, genetically engineered systems, and tumors formed from cell lines implanted into syngeneic animals that vary substantially in their breadth of use, cost, disease latency, and immune physiology. In patient-derived xenografts, a portion of a patient's tumor is introduced into an immune deficient or humanized animal, typically within Matrigel™ (MT), a mouse sarcoma-derived extracellular matrix extract. However, only ˜40% of xenografts form tumors, even in immune deficient animals. Additionally, responses to immunotherapies elicited in humanized mouse xenograft models are often highly variable and do not predict responses seen in patients. Alternatively, genetically engineered models have oncogenic and other genetic information inserted in the animal genome, sometimes in tissue specific loci, to induce tumor formation. A common example is the mouse mammary tumor virus-polyoma middle tumor-antigen (MMTV-PyMT) mouse model in which the polyoma tumor virus is expressed in the mammary fat pad (MFP) to spontaneously form breast tumors. The spontaneous nature of these responses more closely mimics the variable latency and progression of human tumors. However, the rate of tumor development is highly variable, which has the potential to result in disparate tumor-localized immune microenvironments, making testing of immunotherapies exceedingly challenging. Lastly, syngeneic tumor models consist of immortalized tumor cell lines injected into syngeneic, immunocompetent animals. While this latter model class is considered perhaps the least sophisticated, it is by far the most commonly implemented in the immunotherapy field given the ease and rapid nature in which tumor-bearing animals can be generated in varying animal cohorts by synchronous implantation of cancerous cells, the wide availability of transgenic rodents to enable mechanistic testing, and because mice are the lowest phylogenetic species that have human-mimicking immune systems. However, given the low rate of tumor formation when injected cells are suspended in saline alone, syngeneic TNBC models often employ MT to increase the rate of tumor formation. Unfortunately, this approach is plagued by MT's batch-to-batch variation in composition and uncharacterized effects on the recipient animal's immune response. Furthermore, the above listed model classes are limited in their capacity to generate different tumor immune microenvironments without varying the biology of the engrafted tumor cell line, using depleting or modulatory interventions, or use of a genetic knockout system. This is because disease onset and progression are predicated by the host-tumor interaction, which makes the study of the varied microenvironments seen in the tumors of human patients not easily recapitulated in rodent models without changing tumor cell line or animal host. Accelerating and better predicting immunotherapeutic responses in human patient populations are thus severely limited by the gap in the existing repertoire of preclinical tumor models for immunotherapeutic testing.

In this example, well-defined, degradable synthetic hydrogels used in in vivo tissue engineering applications were implemented as a scaffold to reproducibly form tumors from TNBC cell lines in syngeneic mice. Achieving a tumor formation rate of 100% with short latency, formed tumors exhibited low variability in tumor growth profiles and infiltrating immune cell repertoires, attributes favorable for immunotherapeutic drug screening. Furthermore, anti-tumor immune responses that mimic various patterns of immune microenvironments seen in human TNBC tumors were programmed based on hydrogel formulation through the incorporation of cell adhesive peptides within the synthetic polymer network that direct local immune response. Strikingly, microenvironments formed from the same polymer scaffold but directing varying immune microenvironments resulted in disparate responses to ICB and cancer vaccine immunotherapies. These engineered tumor immune microenvironments therefore address the limitations of current tumor models used for drug screening in the immune oncology field to enable immunotherapeutic testing relevant to the variability in tumor-infiltrating lymphocyte profiles seen in human patients.

### Results

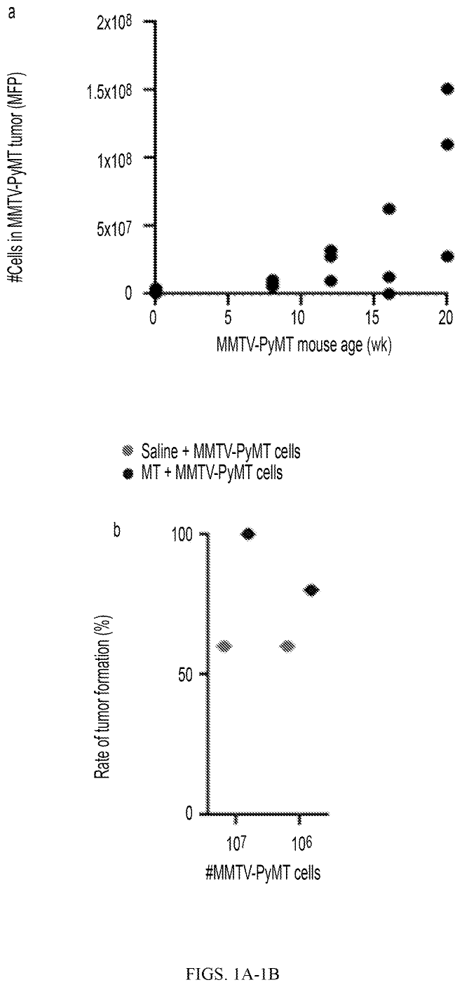

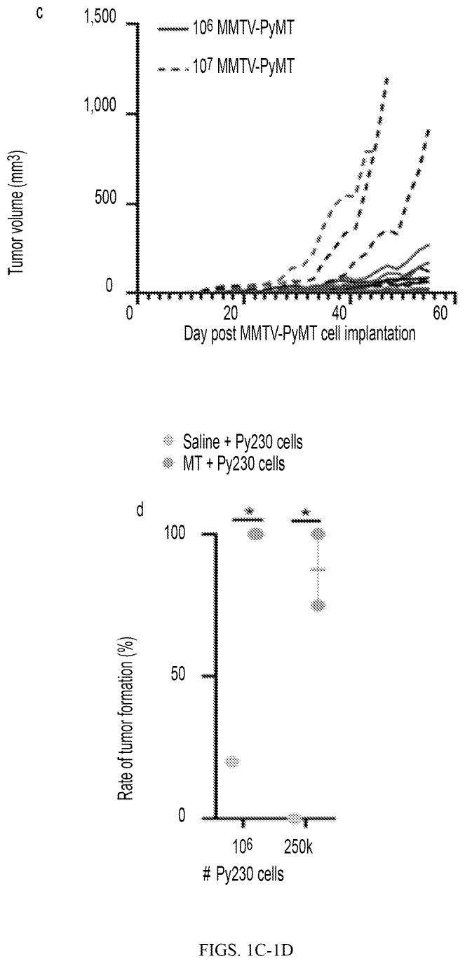

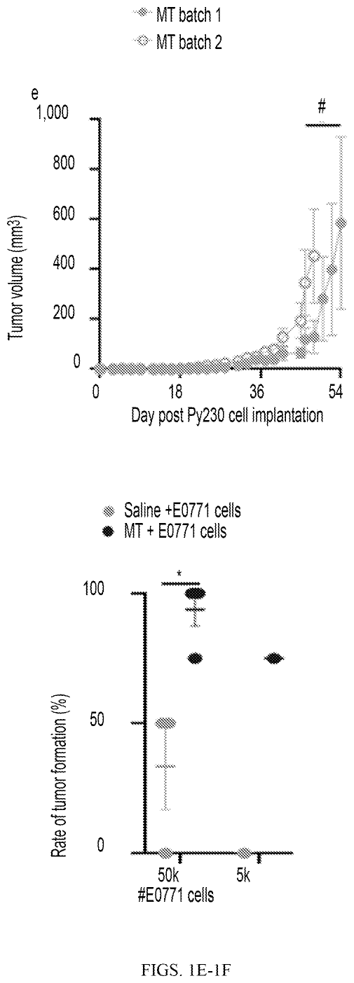

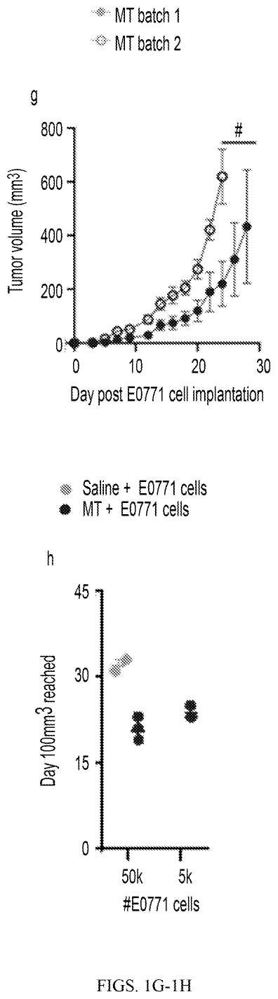

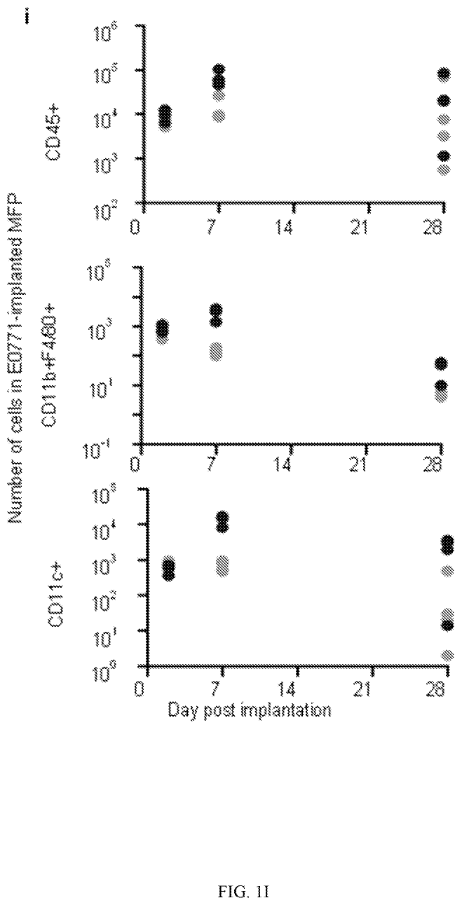

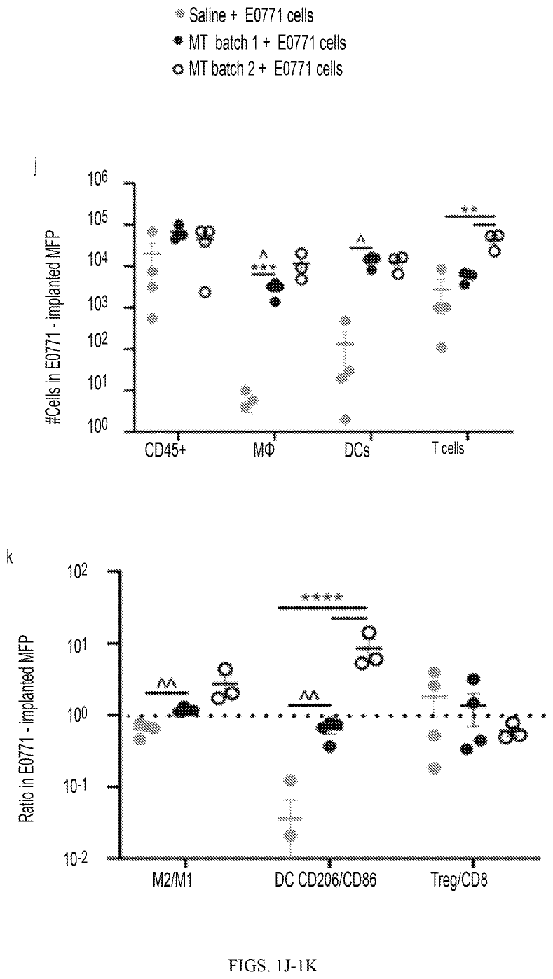

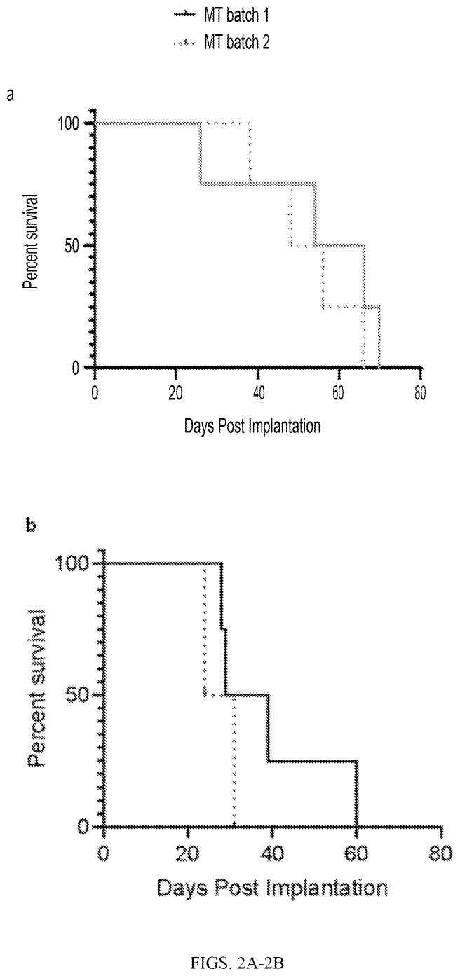

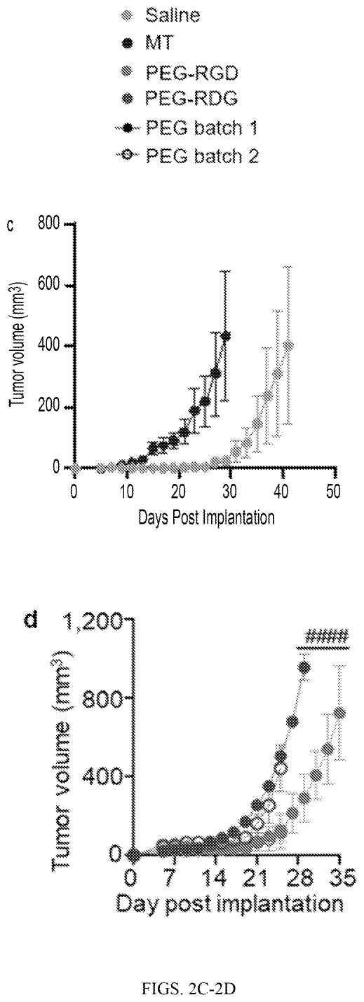

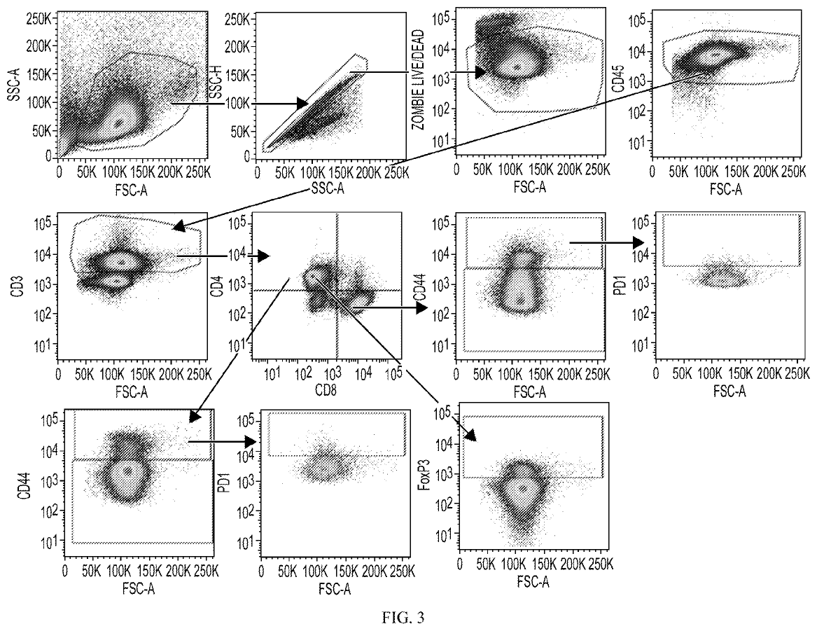

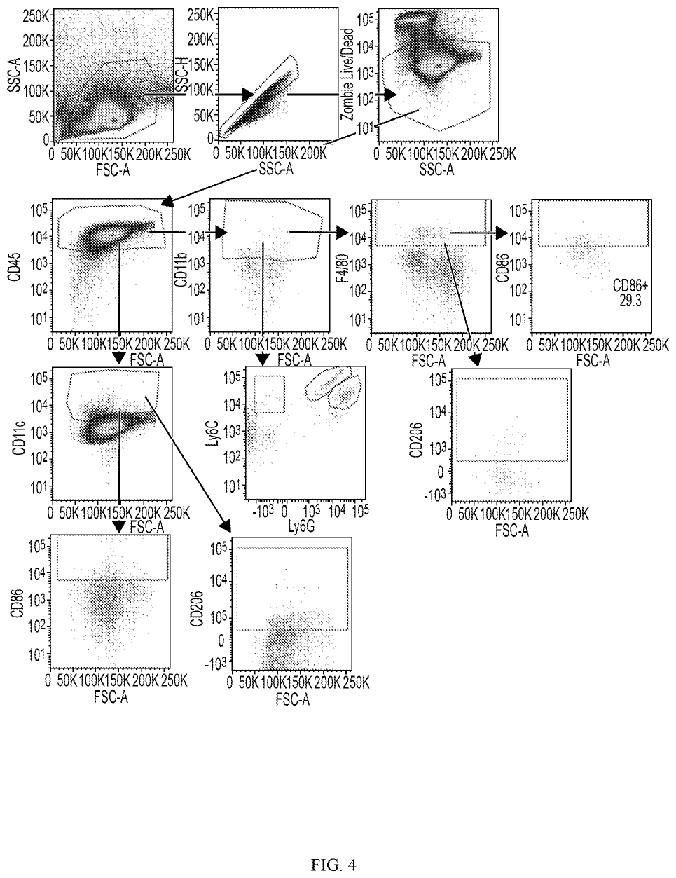

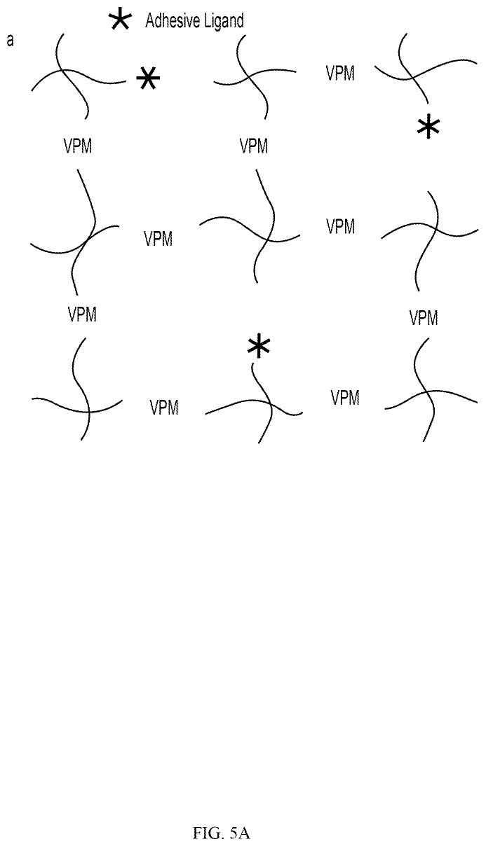

Engineered hydrogel scaffolds for consistent, controllable breast tumor formation in vivo with short latency. Genetically engineered tumor models, including but not limited to that seen in the MMTV-PyMT system, are generally considered the most physiologically relevant systems for the study of immune remodeling in cancer. However, within MMTV-PyMT tumors analyzed at various timepoints throughout disease progression, wide variability was found in terms of the rate of tumor development, with cell numbers ranging by >1 order of magnitude, and coefficients of variation ranging from 26 to 131% (FIG. 1a), which can make immunotherapeutic testing in synchronous animal cohorts exceedingly difficult. Even when excised tumors from lesions formed in MMTV-PyMT mice were dispersed and reimplanted into the MFP of wildtype (non-transgenic, tumor-naïve, immunocompetent) C57/Bl6 mice, rates of tumor formation varied considerably with suspension composition (FIG. 1b). When dispersed in saline alone, implanted cells formed tumors in ˜ 60% of injected animals (FIG. 1b). The use of MT, a commonly used basement membrane extract product generated from mouse sarcomas, increased tumor formation rate, with 100% and ˜85% of injected animals forming tumors with 10⁷ and 10⁶ cells implanted, respectively (FIG. 1b). However, tumor growth was still highly variable between formed tumors (FIG. 1c). Implantation of Py230 cells, a cell line derived from lesions of MMTV-PyMT animals and thought to be less differentiated compared to typical immortalized cell lines, similarly benefited from injection when suspended in MT compared to saline with respect to tumor formation rate, with 100 and ˜90% versus 20% or no tumors forming at 10⁶ versus 0.25×10⁶ cells respectively (FIG. 1d). However, for different batches of MT, the latency of tumor formation from 0.25×10⁶ Py230 cells (FIG. 1e) and animal survival (FIG. 2a) varied considerably. Both trends in MT's benefits with respect to tumor implantation rate and batch effects on tumor growth rate were also seen in the TNBC murine mammary carcinoma line E0771 (FIG. if-h, FIG. 2b-c). Furthermore, the immune microenvironments of E0771 tumors formed in immunocompetent C57/Bl6 mice varied substantially both between animals and matrix vehicle types. Among major immune cell subtypes (total CD45+ cells, macrophages, DCs, and T cells, FIGS. 3-4), the extent of infiltration at day 2 post implantation was initially similar between MT and saline vehicle but subsequently varied substantially at day 7 and 28 post implantation (FIG. 1i). Moreover, tumors implanted in saline exhibited vastly varying extents of infiltration amongst identically treated animals, varying by >2 orders of magnitude between tumors at day 7 post implantation (FIG. 1j). Tumors formed at day 7 after cells were injected in MT resulted in more consistent tumor infiltrating cell levels, varying by less than 0.5 order of magnitude, with coefficients of variation below 50% (relative to >150% in tumors implanted in saline) (FIG. 1j). However, the number of infiltrating T cells was significantly different between different MT batches (FIG. 1j). Macrophage M2/M1 ratios (defined as CD206+ versus CD86+), ratios of CD206 to CD86 expressing DCs, and the ratio of Treg (CD4+CD8−FoxP3+CD25+) to total CD3+CD8+CD4− (CD8) cells also varied substantially amongst mice implanted with tumors using saline (FIG. 1k), an effect ameliorated, save for the case of DC phenotype, by MT as the tumor matrix vehicle (FIG. 1k). Nevertheless, ratios varied substantially between MT batch (FIG. 1k). As a whole, both MT and saline, when used as matrix vehicles for the implantation of breast tumors into immunocompetent animals, induce highly variable immune infiltration, limiting their application for immunotherapeutic testing.

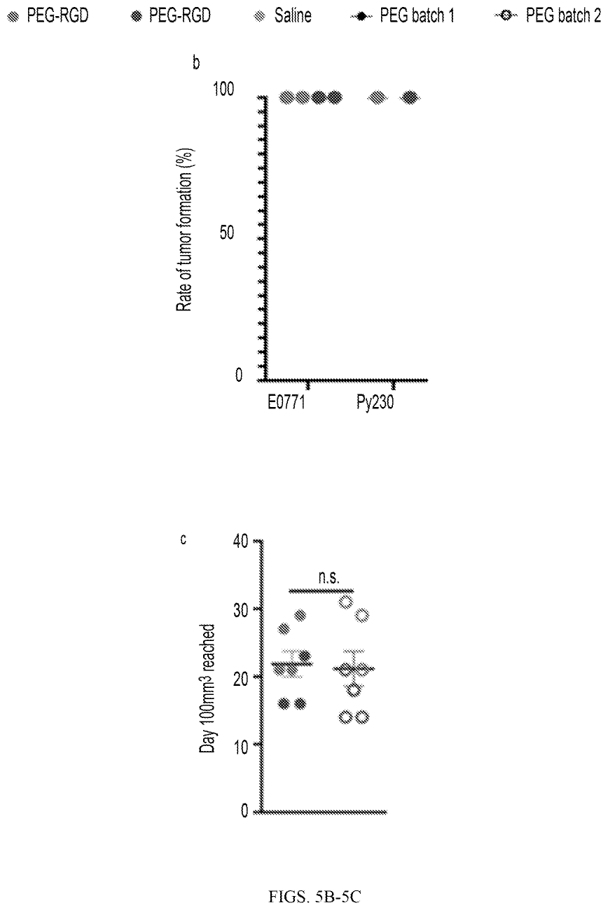

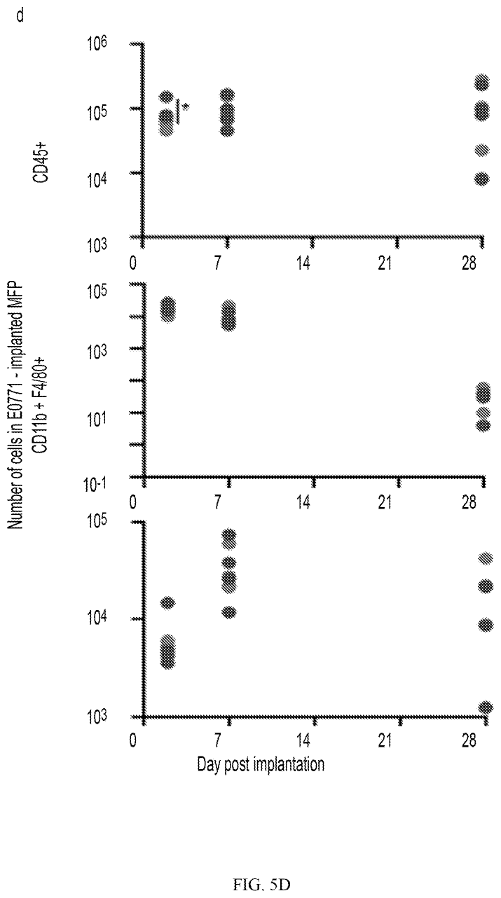

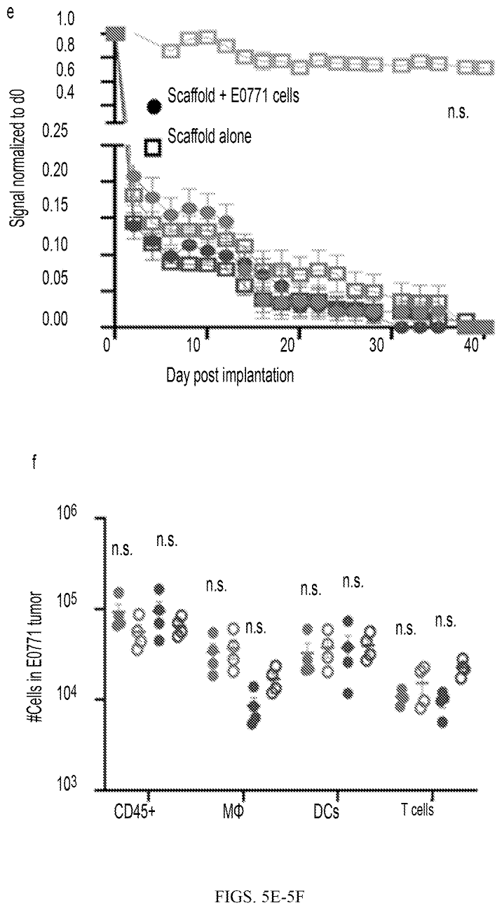

Well-defined synthetic matrices are considered to overcome the deficiencies in tumor latency, growth, and immune infiltration intrinsic to existing preclinical tumor model systems. Specifically, synthetic matrices formed from 4-armed poly(ethylene glycol) (PEG) maleimide (PEG-4MAL) macromers were used. The PEG-4MAL platform provides structurally defined hydrogels with stoichiometric incorporation of biological ligands, improved crosslinking efficiency, and excellent in vitro and in vivo cytocompatibility. To synthesize gels, cysteine-containing RGD adhesive peptide (or its scrambled control peptide RDG) was conjugated to the PEG-4MAL macromer via reaction with the maleimide group to produce a functionalized macromer. This functionalized precursor was then crosslinked into a network in the presence cells by reacting with a bis-cysteine-flanked crosslinker peptide (VPM) susceptible to cleavage by proteases including matrix metalloproteinases (FIG. 5a). In so doing, both Py230 and E0771 cells implanted in these PEG gels induced tumors in 100% of animals (FIG. 5b) at total implanted cell amounts of 0.25×10⁶ and 0.05×10⁶ cells, respectively, where low tumor formation rates were seen for saline and variable for MT at similar, or higher, cell amounts of 0.25-1×10⁶ or 1-10×10⁶ Py230 or E0771 cells, respectively (FIG. 1f). Likewise, the time to reach 100 mm³ in tumor volume, a size at which E0771 tumors grow exponentially, was consistent between batches of PEG scaffolds (FIG. 5c). This high success of tumor formation was not dependent on adhesive peptide as the non-adhesive RDG peptide also yielded 100% tumor formation (FIG. 5b). Total leukocyte infiltration was higher into PEG-RGD tumors at d2 after implantation, but otherwise, overall infiltration of leukocytes, macrophages, and DCs were equivalent between hydrogels presenting RGD and RDG over 28 d (FIG. 5d). The in vivo scaffold degradation was examined using near infrared dye-labeled hydrogels and IVIS and no difference in matrix vehicle signal loss was observed with inclusion of tumor cells compared to the tumor-cell free matrix vehicle, nor between PEG gels presenting RGD and RDG peptides (FIG. 5e). In contrast to that seen in MT, tumor growth rate as well as immune infiltration into tumors at day 7 post implantation by injection in PEG hydrogels were highly consistent between polymer batch (FIG. 5f and FIG. 2d), despite the polymer mixtures being prepared >1 year apart and high degrees of variability in infiltration levels into tumors formed in saline or MT at this tumor stage (FIG. 1i-k). This engineered hydrogel system enables the consistent and reproducible growth of tumors formed from TNBC cells that are immunologically consistent for in vivo disease modeling.

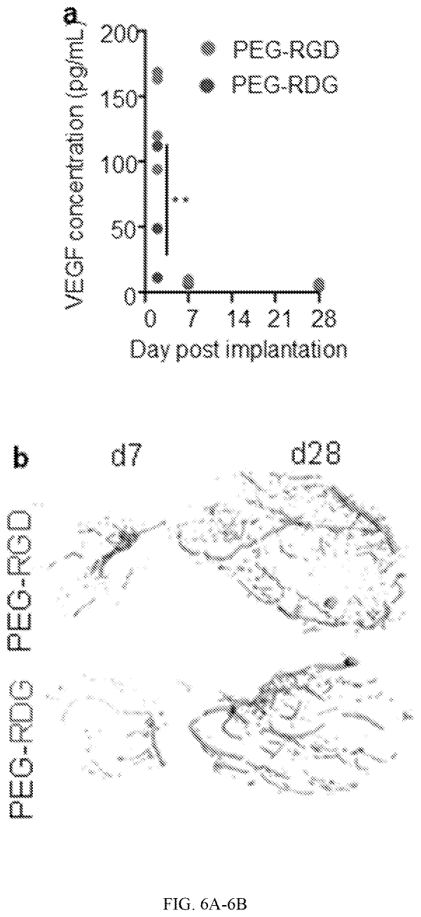

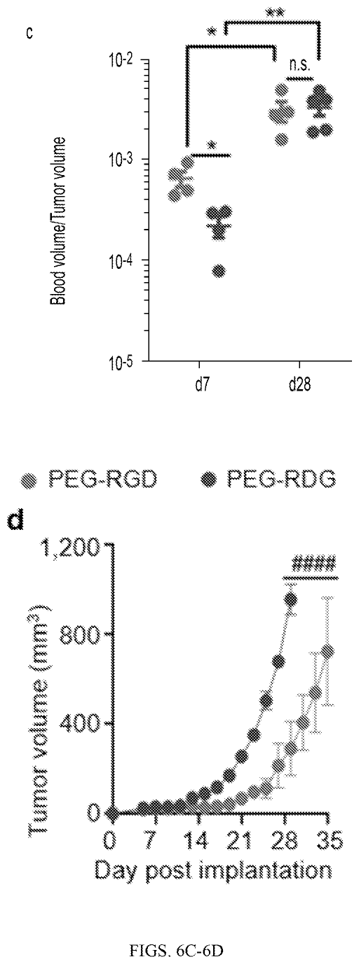

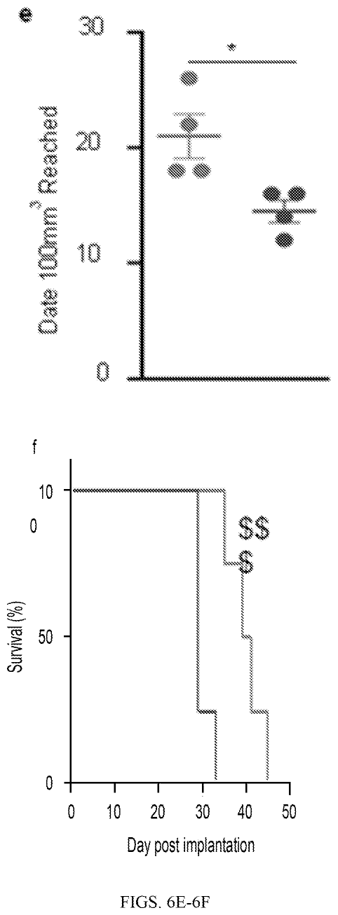

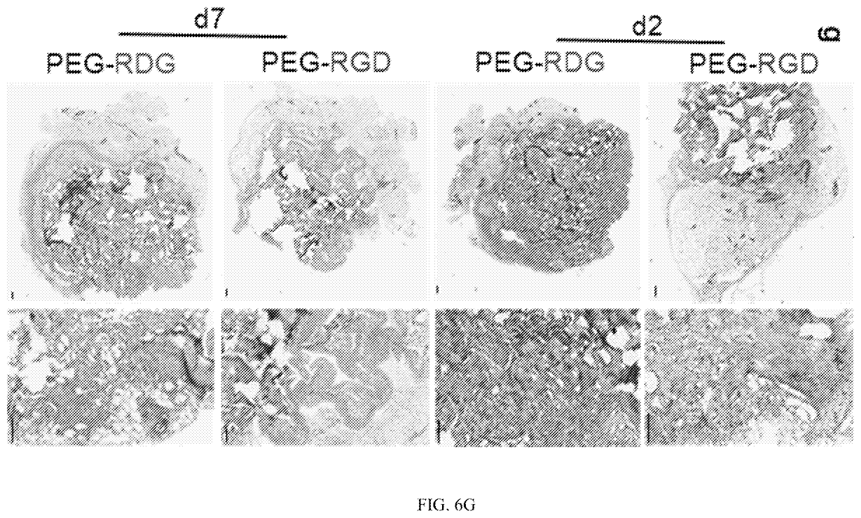

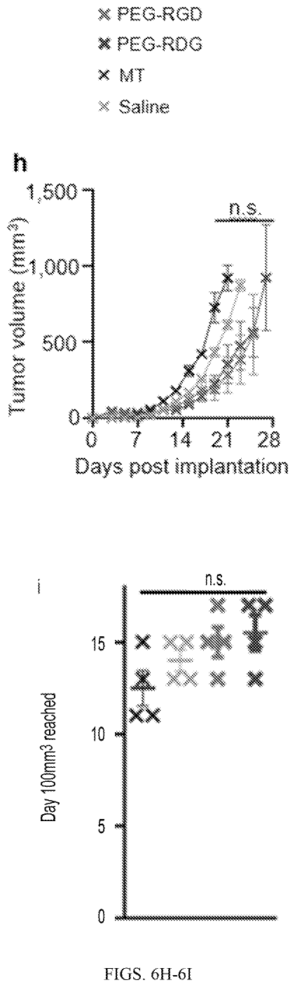

Hydrogel adhesive peptide directs immune infiltration into breast tumor microenvironments. Given the potential for adhesive ligands such as RGD to impact vascularization within scaffolds, the vascularization response of tumors formed from different hydrogel formulations was also investigated. First, vascular endothelial growth factor (VEGF)-A levels within E0771 tumors implanted within PEG gels differed substantially based on adhesive ligand at early but not later tumor stages (FIG. 6a). Tumors formed from E0771 cells in RGD-functionalized PEG hydrogels (PEG-RGD) matrices also exhibited higher degrees of vascularization compared to RDG-functionalized PEG hydrogels (PEG-RDG) tumors as measured by microcomputed tomography (CT) of Microfil-perfused animals at early (d7) but not later (d28) tumor stages (FIG. 6b-c). Nevertheless, tumor growth rate did not reflect the expected effect of tumor vascularization induced by adhesive ligand incorporation into the scaffold, as PEG-RDG tumors grew more rapidly than PEG-RGD tumors. Notably, PEG-RDG tumors exhibited faster tumor growth, decreased time to 100 mm³, and shorter survival (FIG. 6d-f). Histological analysis of these tumors revealed that PEG matrix vehicles remained largely intact at day 2 post tumor implantation and by day 7 had more tissue integration (FIG. 6g). Qualitatively, PEG-RDG tumors also contained higher cell densities compared to PEG-RGD tumors (FIG. 6g). In order to delineate the immunological and structural effects of tumor implantation within a scaffold, E0771 tumors were implanted within saline, MT, and both engineered hydrogels, into NOD-SCID-gamma (NSG) mice (FIG. 6h-i), which are immune deficient in B cells, T cells, and natural killer cells. This revealed that differences in tumor latency and growth rate between scaffold type were lost. This striking result shows that the immune competency of the host, and as a result, infiltration by host immune cells into the tumor cell-laden scaffold, impacts scaffold-dependent effects on tumor formation.

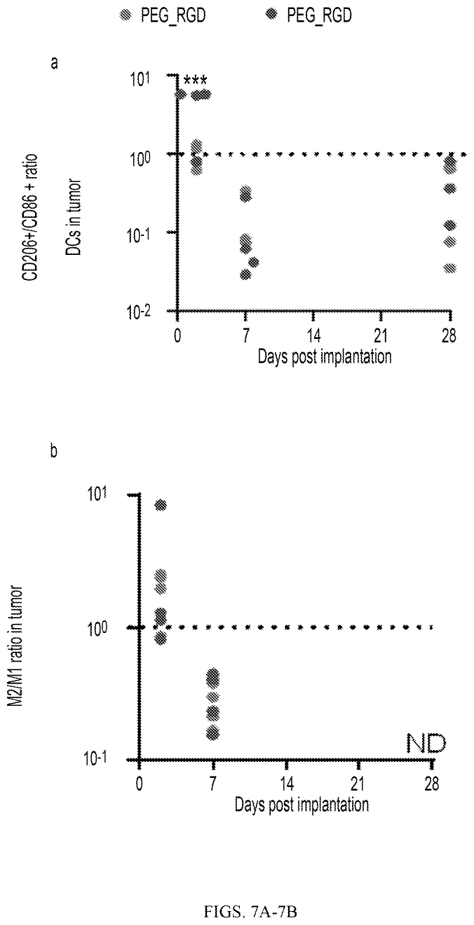

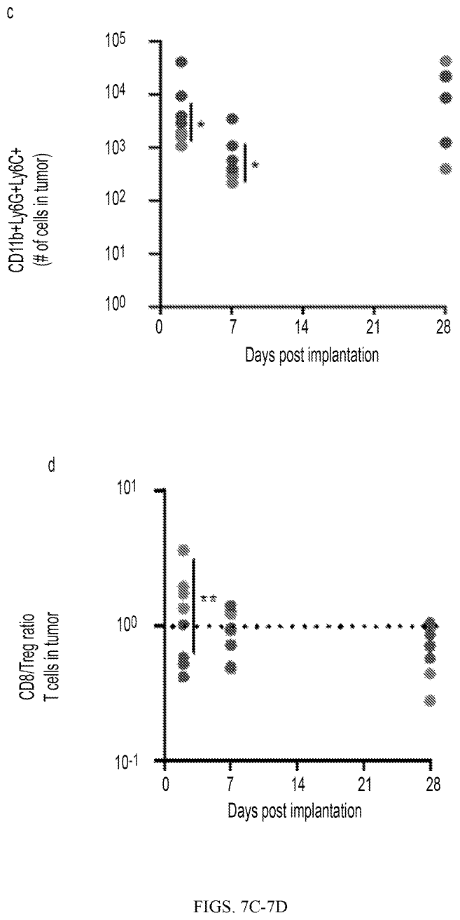

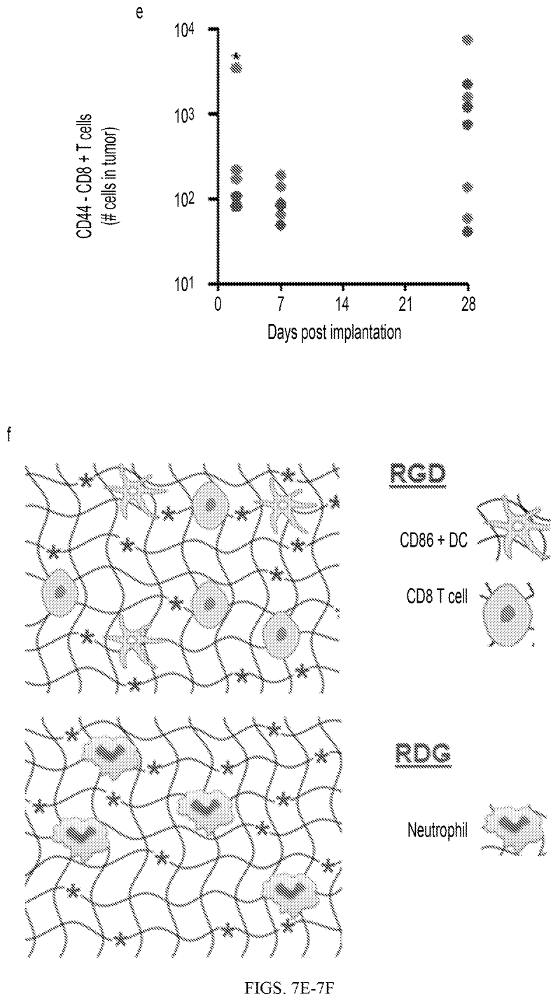

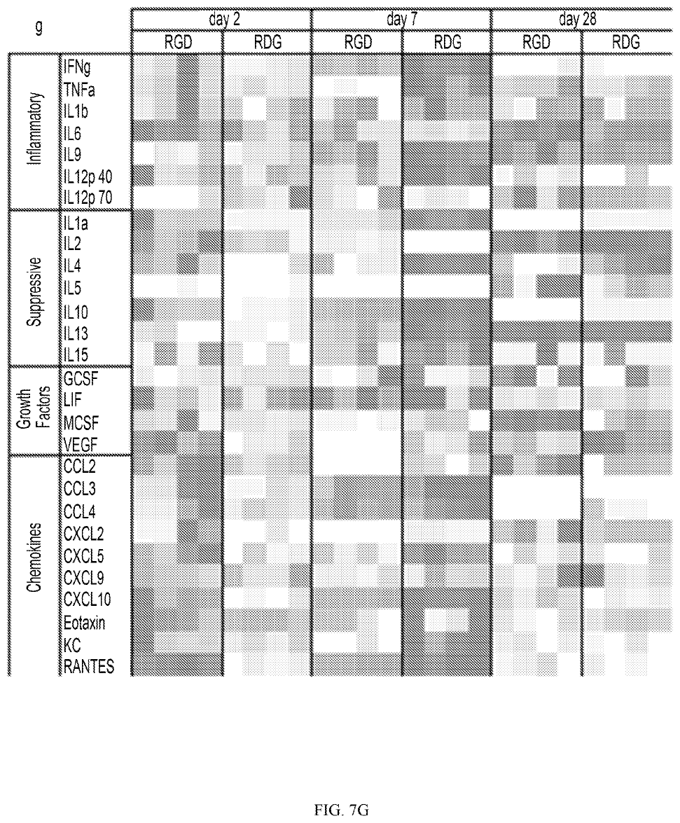

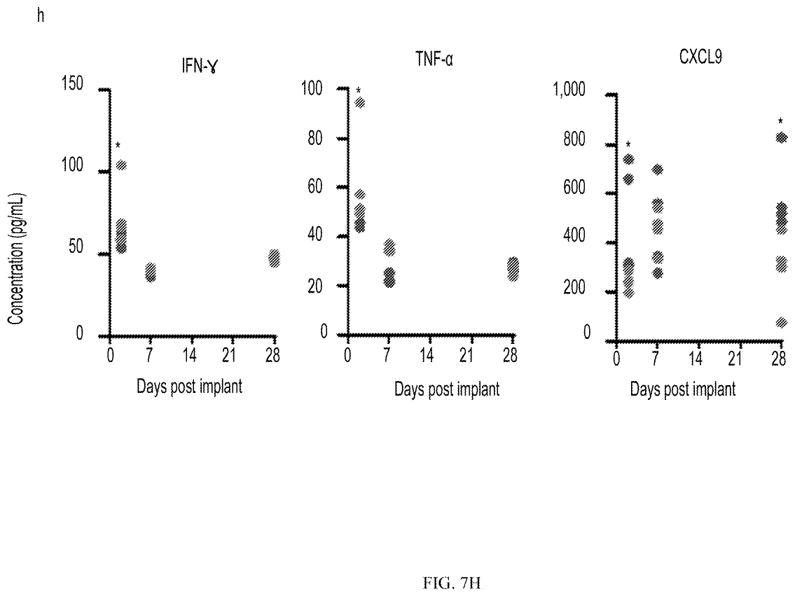

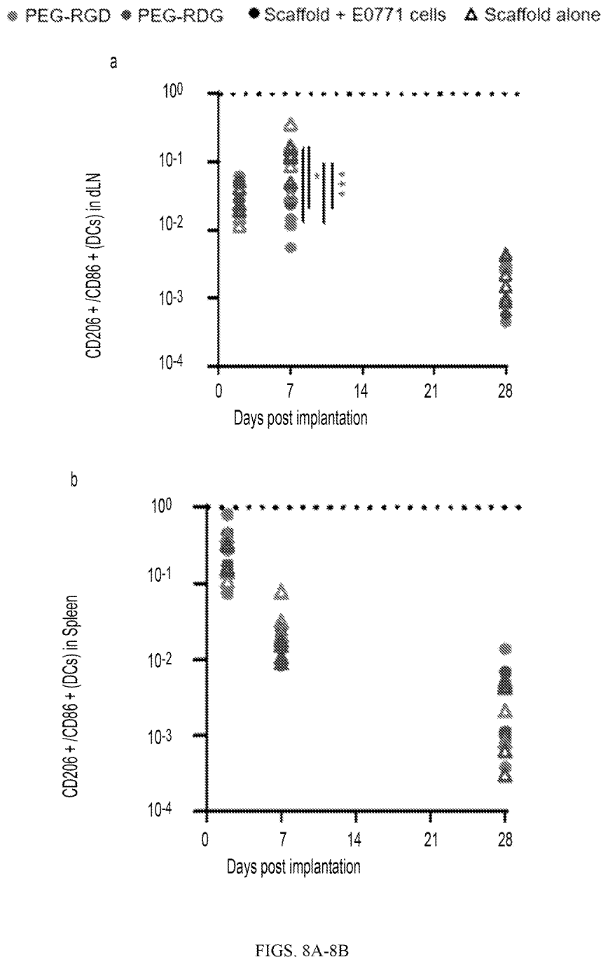

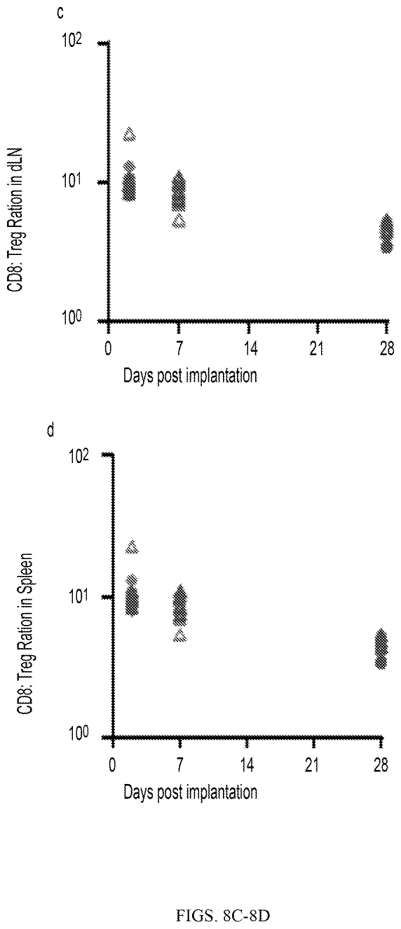

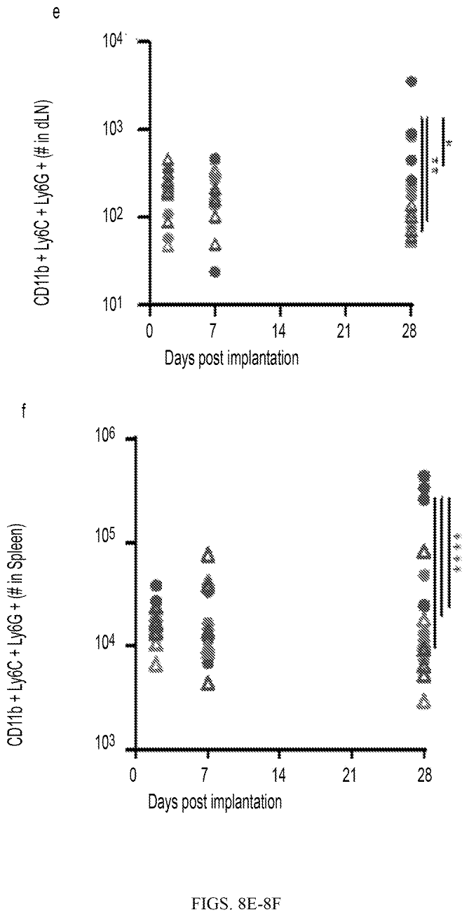

To interrogate the apparent immune association of differences in tumor latency and growth rate directed by adhesive ligand incorporation into the matrix vehicle injected with E0771 cells, immune phenotyping of formed breast tumors during development and growth was performed, focusing on cell subsets previously implicated in tumor latency and disease progression. In particular, DCs within tumors were found to exhibit higher activation states when PEG-RGD compared to PEG-RDG scaffolds were used at early times post implantation (d 2), an effect lost at later (d 7 and 28) tumor stages (FIG. 7a). Whereas M2/M1 ratios were similar at all measured tumor stages (FIG. 7b), infiltration of CD11b+Ly6C+Ly6G+ cells, a cell type that in a cancer context is likely a neutrophil subset, into tumors formed from PEG-RGD was higher to that of tumors formed with PEG-RDG at day 2 and 7 but not at day 28 post implantation (FIG. 7c). Tumors formed from PEG-RGD scaffolds also exhibited higher ratios of CD8+ to regulatory T cells compared to tumors formed from PEG-RDG at day 2 post implantation, a difference that, like DC phenotype, was not seen at day 7 and 28 post implantation (FIG. 7d). CD44−, naïve, CD8+ T cells were higher in day 2 tumors in PEG-RGD tumors, and this difference similarly dissipated in later stage tumors (FIG. 7e). As a whole, on a cellular basis, PEG-RGD tumors induced higher infiltration of CD8+ T cell and CD86+ DCs, while PEG-RDG tumors contained more neutrophils (FIG. 7f). No differences were noted in immune cell contents of the spleen and draining lymph node, except for higher numbers of CD11b+Ly6C+Ly6G+ in spleens at day 28 post implantation of E0771 cells within PEG-RDG scaffolds (FIG. 8). Together, these results demonstrate that engineered hydrogels modulate immune infiltration locally throughout tumor development and progression.

Cytokine production within these tumors was also assessed across time. As a whole, higher levels of cytokines were present within PEG-RGD tumors relative to PEG-RDG tumors at day 2 and 7 post tumor implantation, with the exception of C-X-C motif chemokine ligand (CXCL) 9, an effect that dissipated by day 28. In particular, concentrations of interferon (IFN) γ and tumor necrosis factor (TNF) α, cytokines associated with a Th1 response, were enhanced at day 2 in PEG-RGD tumors relative to PEG-RDG tumors (FIG. 7h) showing that changes in local immune cells discussed above (FIG. 7f) induce functional cytokine responses within the formed tumors. Similarly, interleukin (IL) 1a, IL4, IL10, IL13, CXCL10, and RANTES were elevated in PEG-RGD tumors at d2; and IL6, IL13, KC, and RANTES were elevated in PEG-RGD tumors at d7 (FIG. 7g, Table 1), showing the overall increase in inflammatory response. CXCL9, a chemokine which can be attributed to neutrophils, showed opposite trends compared to all other cytokines assessed here (FIG. 7g-7h), however, with higher concentrations in PEG-RDG tumors relative to PEG-RGD tumors at day 2 and 28 of tumors growth, showing neutrophil infiltration as being functional. These cytokine responses correlate with cellular responses above, with PEG-RGD tumors inducing a CD8+ T cell and CD86+DC, Th1 type response, while PEG-RDG tumors induce a neutrophil-driven response. Of note, human TNBC tumors have been shown develop one of three immune signatures: immune-infiltrated responses driven by neutrophils; immune-infiltrated tumors driven by M1 macrophages and CD8+ T cells; and immunologically “cold” tumors, generally devoid of immune cells. The two immune-infiltrated tumor subsets relevant to human TNBC appear recapitulated using the engineered scaffold system presented here, with PEG-RGD tumors demonstrating a CD8+ T cell-driven response, and PEG-RDG tumors demonstrating a neutrophil-driven response (FIG. 7f).

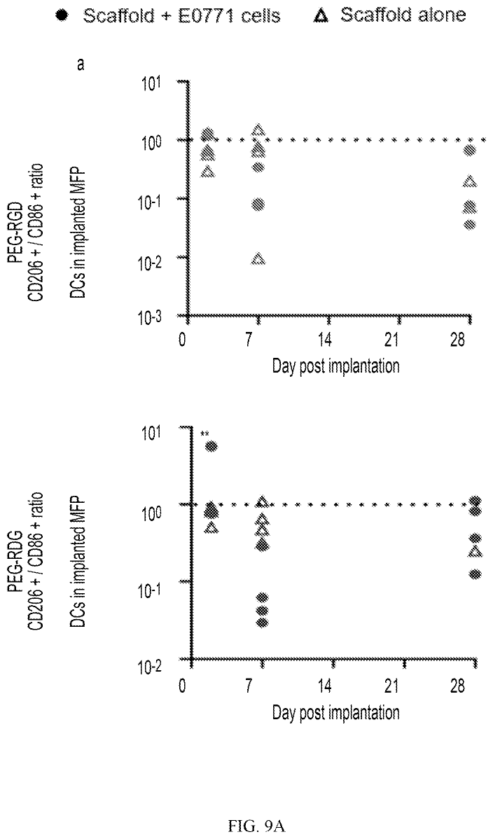

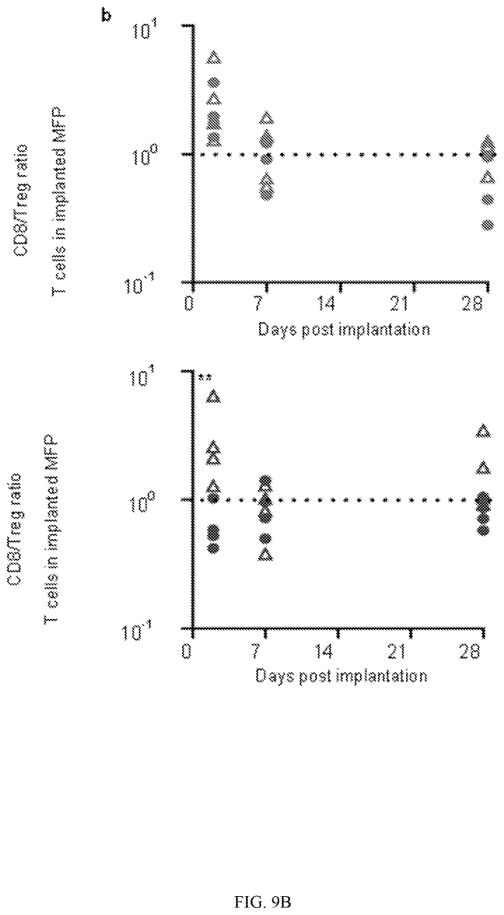

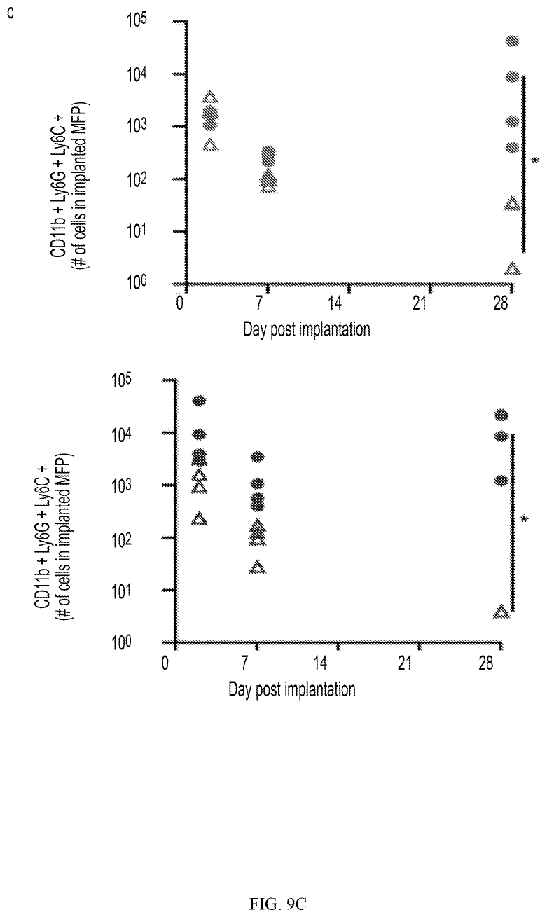

As both the tumor cells and adhesive ligands tethered to the hydrogel alter the immune status of developing tumors, the infiltrating immune cells were compared in scaffolds implanted with or without tumor cells. The phenotype of DCs (CD206:CD86 ratio) and type of T cell (by CD8+ T cell: Treg ratio), which defined the cellular phenotype within PEG-RGD tumors (FIG. 7f), were identical within PEG-RGD scaffolds implanted with or without tumor cells, showing that presentation of RGD within the scaffold (i.e., not the cancer cells) drove this Th1 response (FIG. 9a-b). However, within PEG-RDG scaffolds, both DC phenotype and type of T cell were initially (at day 2) higher in tumor cell-containing scaffolds compared to scaffold vehicles implanted alone before equalizing at day 7 and day 28 (FIG. 9a-b), showing that the initial Th1 response in PEG-RDG scaffolds were tumor cell- and not adhesive ligand-driven. For neutrophils, which infiltrated PEG-RDG scaffolds to greater extents, initial infiltration (at d 2) was not different between scaffolds implanted with or without tumor cells when implanted with hydrogels presenting either RGD or RDG (FIG. 9c). However, at day 7 and 28, scaffolds implanted with tumor cells had higher neutrophil infiltration showing a primarily tumor driven response (FIG. 9c). As a whole, the Th1 response in PEG-RGD scaffolds appears associated with the adhesive ligand engineered within the system. Contrastingly, the neutrophil response in PEG-RDG scaffolds was tumor cell-driven. Thus, scaffold-directed host immune response within the tumor, but not tumor vascularization, correlate with tumor growth rate. Taken together, these results show that the engineered delivery scaffold for the tumor cells can direct the local immune microenvironment within the tumor.

Matrix scaffold-directed tumor immunophenotype dictates immunotherapeutic responses. The response of tumors formed from TNBC E0771 cells implanted within different matrix vehicles to immunotherapy was assessed. Immunotherapies evaluated included a in situ cancer vaccine, in the form of intratumorally (i.t.) injected Toll-like receptor 9 agonist CpG, and ICB in the form of intraperitoneally (i.p.) administered monoclonal antibodies (mAb) recognizing cytotoxic T lymphocyte antigen (CTLA) 4 and PD1. Responses to isotype control mAb also administered i.p. were also evaluated. Therapies were administered to individual animals once tumors reached 100 mm³ in ellipsoidal volume in order to account for the highly variable differences in tumor formation and growth rate between matrix vehicle types.

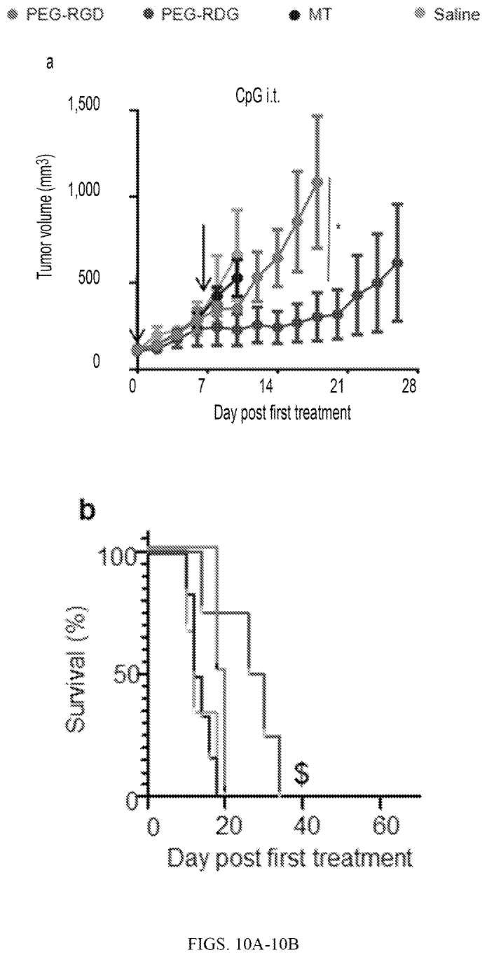

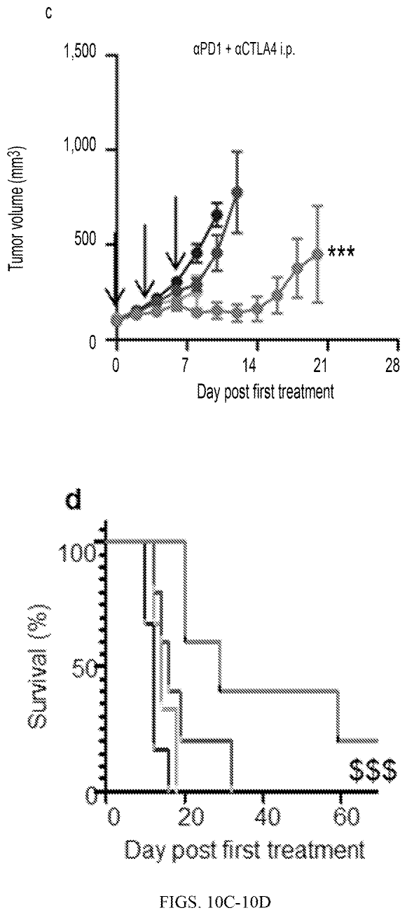

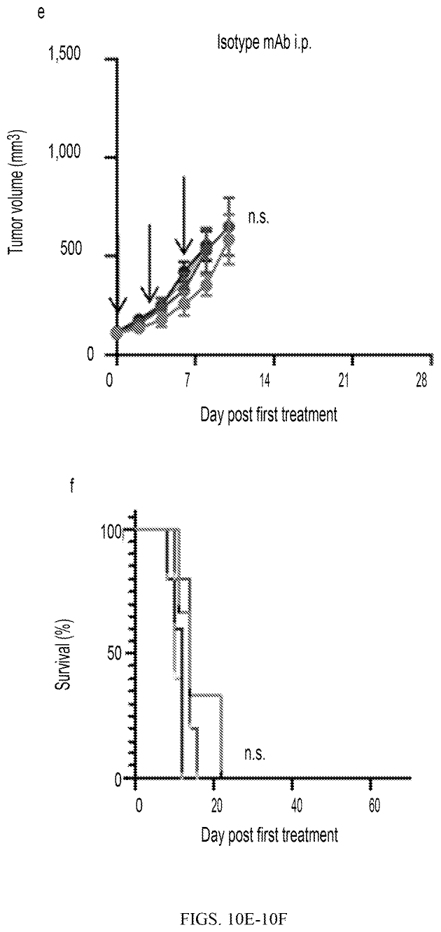

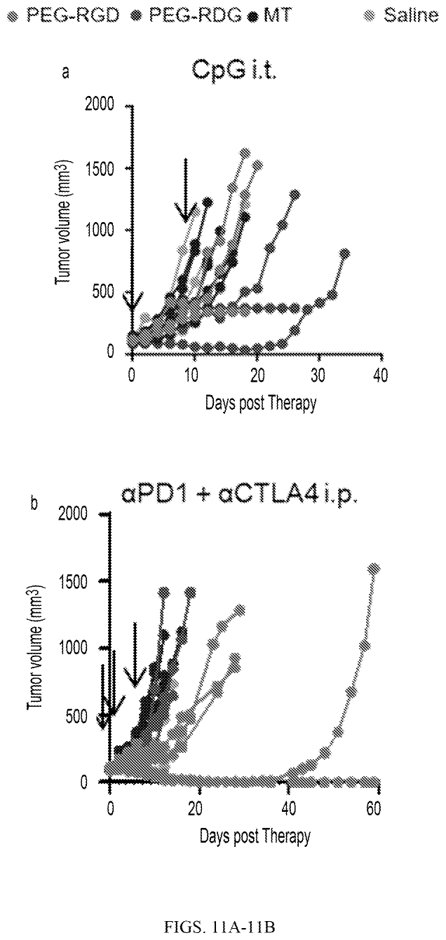

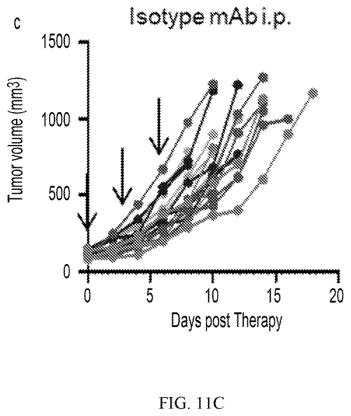

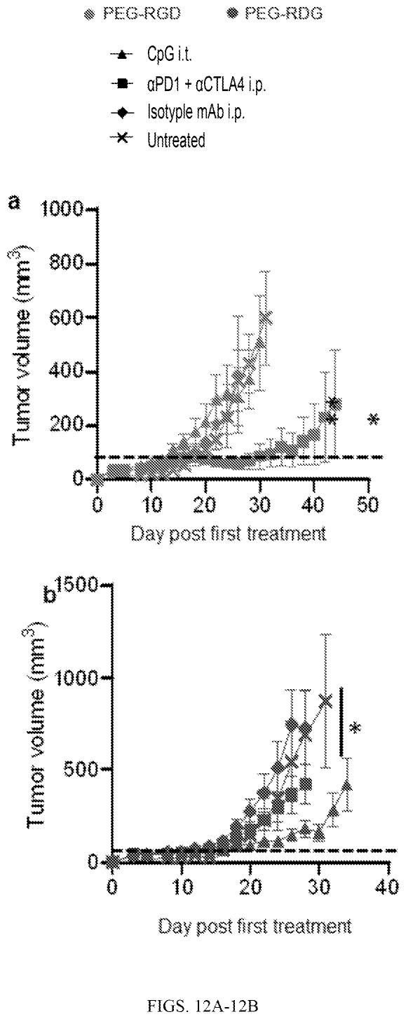

With i.t. CpG treatment, tumors implanted in saline, MT, and PEG-RGD matrices did not respond to the it. vaccine, demonstrating similar growth curves as the untreated animals (FIG. 10a and FIG. 11-12). In contrast, tumors implanted using PEG-RDG scaffold exhibited significant slowing of tumor growth (FIG. 10a) that resulted in prolonged animal survival as a result of i.t. vaccination (FIG. 10b). These results are in good agreement with an activation of the Th1 response previously poor in these tumors (FIG. 7f). Contrastingly, after ICB treatment, therapeutic responses were minimal in mice bearing tumors formed from PEG-RDG, saline, and MT matrices, but robust in animals formed from PEG-RGD matrices (FIG. 10c, FIG. 11-12). As a result, the overall survival of PEG-RGD tumor-bearing animals was prolonged relative to all other groups (FIG. 10d). This result shows that ICB therapy is beneficial in the context of tumor immune microenvironments that have a heightened local CD8+ T cell and CD86+DC response. Importantly, no matrix vehicle-specific differences in tumor growth or survival were observed for animals receiving isotype control mAb (FIG. 10e-f), nor were survival benefits that varied by therapy class associated with tumor growth rates in untreated animals (FIG. 12). These results demonstrate that local immune microenvironments within the tumor programmed by the engineered scaffold direct the resulting sensitivity versus resistance to differing classes of immunotherapy.

### DISCUSSION

The demand for a modernized immune oncology drug development testing pipeline has mushroomed with the advent of the cancer immunotherapy era. Such improvements include radical advancements in in vitro bioassays and organoid technologies. Yet despite their attractiveness with respect to cost, speed, scale, and mechanistic insights, such approaches necessarily oversimplify the spatial and temporal complexity of the adaptive immune response in cancer and are unable to predict therapeutic benefit. Seemingly in complete opposition to this, there is a push within the in vivo disease modelling space for the use of increasingly complex genetic models with the goal of better recapitulating human disease. Despite their advantages in modeling the heterogeneity of human cancer and adaptive immune response, however, these systems are prohibitively expensive, highly variable, slow, and as a result poorly scalable. An ideal platform for screening the therapeutic efficacy of immune oncology drugs are the tumor immune microenvironments that can be generated with deterministic reproducibility at scale that do not sacrifice the dynamic complexity of an in vivo adaptive immune response.

Here, a robust and scalable platform is described for the in vivo modeling of immunologically defined tumors inspired by but improving upon existing syngeneic rodent tumor models. In so doing, this system ameliorates the limitations with patient-derived xenografts, as it is much less expensive and tumors form 100% of the time across multiple independent experiments; genetically engineered mouse models, as both the number of tumors and immune responses against those tumors are consistent and controllable; and current syngeneic models, as the immune response is consistent between batches and can be modulated to more closely mimic different immune responses observed in clinical samples.

The concept of immunologically defined tumors that are programmable based on scaffold composition offers numerous advantages with respect to tumor immunotherapy drug development. Such an outcome has been demonstrated here with growth trajectories being shaped by the local immune microenvironment. More provocatively, immunotherapeutic sensitivity versus resistance vary with therapy class and scaffold composition, without changing the tumor cell used that changes the underlying biology. As this benefit is afforded by the modular nature of the material system, tethering other adhesive ligands or immunomodulatory agents to the scaffold to further direct immune responses against the developing tumor is an option. Additionally, implementing this scaffold for other solid tumor types for which current immunotherapeutic strategies are lacking, in order to better predict immune responses and develop novel, more effective anti-cancer treatments, can be considered. The concept of a tissue engineered tumor thus resembles strategies that have long been explored in the context of regenerative engineering and in in vitro screening and organoid systems, but heretofore have been underutilized for in vivo tumor immunotherapy drug development.

### Materials and Methods

Cell culture. E0771 breast cancer cells, derived from C57/Bl6 mice, were cultured in Dulbecco's modified eagle's medium (Gibco, Thermo Fisher Scientific, Inc., Waltham, MA) with 10% heat-inactivated fetal bovine serum (Gibco, Thermo Fisher Scientific, Inc.) and 1% penicillin/streptomycin/amphotericin B (Life Technologies, Carlsbad, CA). Py230 murine mammary tumor cells were cultured in F-12K medium (Corning, VWR International, Inc.) with 5% heat-inactivated fetal bovine serum (Gibco, Thermo Fisher Scientific Inc.), 1% penicillin/streptomycin/amphotericin B (Life Technologies), and 0.1% MITO+ serum extender (Corning, VWR International, Inc.). Cells were maintained at 37° C. and 5% CO2 and passaged at ˜70-80% confluency using 0.05% (E0771) or 0.25% (Py230) Trypsin-EDTA (Thermo Fisher Scientific, Inc.).

Animal tumor models. All protocols were approved by Georgia Tech's Institutional Animal Care and Use Committee. C57/Bl6 or Nod-Scid-Gamma (NSG) mice were purchased at 6 wk of age from Jackson Laboratory (Bar Harbor, ME). MMTV-PyMT mice backcrossed onto C57/Bl6 background were bred in house. MMTV-PyMT mice were monitored on a weekly basis throughout tumor development and progression. For MMTV-PyMT transplant studies, tumors were excised around 200 mm³ in ellipsoid volume. MMTV-PyMT tumors were separated using 18G needles and incubated with 1 mg/mL collagenase D (Sigma Aldrich) in Dulbecco's phosphate-buffered saline (D-PBS) for 60 min at 37° C. with 5% CO2. Tumors were then dissociated by pushing through a 70 μm cell strainer (Greiner Bio-One, Monroe, NC) twice and washed with D-PBS, and counted for implantation. 5-500×10³ E0771 cells, 0.25-1×10⁶ Py230 cells, or 1-10×10⁶ PyMT tumor cells in 30 μL of appropriate scaffold were injected in the fourth (inguinal) mammary fatpad. Animals were monitored every 1-3 days during tumor growth. Tumor dimensions were measured with calipers in three dimensions and reported as ellipsoidal volume. Animals were euthanized if they displayed signs of rodent illness (weight loss >10%, hunched, ungroomed appearance) or if the tumor reached 15 mm in any dimension.

Flow cytometry. Tumor, lymph node, and spleen samples were excised from animals after CO2 asphyxiation. Tumor samples were broken up using 18G needles and incubated with 1 mg/mL collagenase D (Sigma Aldrich) in D-PBS for 4 h at 37° C. LN samples were incubated with 1 mg/mL collagenase D (Sigma Aldrich) in D-PBS for 75 min at 37° C. Following collagenase incubation, samples were pushed through 70 μm cell strainers (Greiner Bio-One), washed with D-PBS, pelleted, and plated at appropriate dilutions in a 96-well U-bottom plate (VWR International, Inc.). Spleen capsules were disrupted using 18G needles, pushed through 70 m cell strainers (Greiner Bio-One), washing with D-PBS, pelleted, and resuspended in 1 mL red blood cell lysis buffer (Sigma Aldrich) for 7 min at room temperature. Samples were quenched with ˜35 mL D-PBS, pelleted, and plated at appropriate dilutions. Cells were blocked with CD16/CD32 antibody (clone 2.4G2, Tonbo Biosciences, San Diego, CA) for 5 min on ice, washed, and stained with a fixable viability dye Zombie Aqua (1:100 dilution, Biolegend, Inc.) for 30 min at room temperature, before quenching with 0.1% bovine serum albumin in D-PBS (flow cytometry buffer). Antibodies were obtained from Biolegend, Inc. unless otherwise specified, and prepared at the following dilutions on the basis of preliminary titrations: APC-Cy7 anti-mouse CD45 (0.625:100), AF700 anti-mouse CD11b (1.25:100), BV605 anti-mouse CD64 (2.5:100), BV711 anti-mouse Ly6C (2.5:100), FITC anti-mouse MerTK (1:100), PerCP anti-mouse Ly6G (eBioscience, Thermo Fisher Scientific, Inc.; 2.5:100), PE-Cy7 anti-mouse CD11c (1.25:100), BV421 anti-mouse MHC-II (1.25:100), PE anti-mouse CD86 (5:100), and BV786 anti-mouse F4/80 (2.5:100) for APC panel; or PerCP anti-mouse CD45 (0.625:100), BV711 anti-mouse CD3 (1.25:100), APC-Cy7 anti-mouse CD4 (0.15625:100), FITC anti-mouse CD8 (0.3125:100), BV786 anti-mouse PD1 (1.25:100), AF700 anti-mouse CD25 (1:100), and BV421 anti-mouse CD44 (5:100) for T cell panel. APC panel samples were then washed and incubated in IC fixation buffer (eBioscience, Thermo Fisher Scientific, Inc.) for 60 min at room temperature in the dark. Cells were then incubated with APC anti-mouse CD206 (2.5:100) in IC permeabilization buffer (eBioscience, Thermo Fisher Scientific, Inc.) for 60 min at room temperature in the dark. T cell panel samples were washed and resuspended in FoxP3/Transcription factor fixation/permeablization solution (eBioscience, Thermo Fisher Scientific, Inc.) for 60 min on ice in the dark. Cells were then incubated with PE anti-mouse FoxP3 (5:100) in FoxP3/Transcription factor fixation/permeabilization buffer (eBioscience, Thermo Fisher Scientific, Inc.) for 75 min on ice in the dark. Both APC and T cell panel samples were resuspended in flow cytometry buffer and kept at 4° C. for a maximum of 48 h before analysis using a customized BD LSRFortessa (BD Biosciences). Compensation was performed using ArC (for live/dead) or UltraComp (for antibodies) compensation beads (Thermo Fisher Scientific, Inc.) and data analyzed using FlowJo software version 10 (FlowJo LLC, Ashland, OR).

PEG hydrogels. PEG-4MAL hydrogels were prepared as described previously. Briefly, PEG-4MAL macromer (molecular mass of 22,000 Da; Laysan Bio, Inc., Arab, AL) was dissolved in 4-(2-hydroxyethyl)piperazine-1-ethanesulfonic acis (HEPES) buffer (10 mM in D-PBS, pH 7.4) at 15% w/v (2.5× macromer density, for a final 6% w/v PEG-4MAL concentration). Cell adhesive and crosslinking peptides were custom synthesized by GenScript Biotech (Piscataway, NJ). Cell adhesive peptide RGD (GRGDSPC (SEQ ID NO: 1)) and its scrambled control RDG (GRDGSPC (SEQ ID NO: 2)) were dissolved in HEPES buffer at 5.0 mM (5× ligand density, for a final 1.0 mM ligand concentration and mixed with PEG-4MAL at a 2:1 PEG-4MAL/ligand ratio to generate functionalized PEG-4MAL precursor. Bis-cysteine crosslinking peptide (GCRDVPMSMRGGDRCG (SEQ ID NO: 3)) was dissolved in HEPES at a density corresponding to 1:1 maleimide to cysteine ratio after accounting for maleimide groups reacted with adhesive peptide. Cells were resuspended at 5× final density in sterile saline and kept on ice. A final density of 50,000 cells were encapsulated in all hydrogels. For all the examples listed, the engineered synthetic hydrogel formulation used was 6% w/v PEG-4MAL functionalized with 1 mM RGD or RDG and crosslinked with VPM (storage modulus, G′=300 Pa). The functionalized PEG-4MAL (PEG-4MAL:adhesive peptide) and cell mixture was combined with the crosslinker at 2:1:1:1 volume ratio (PEG-4MAL:adhesive peptide:cells:crosslinker) immediately before injection. For in vivo hydrogel degradation experiments, both RGD and RDG were conjugated with AlexaFluor750 dye through NHS ester reaction kit following manufacturer instructions (A37575, Molecular Probes by Life Technology).

IVIS imaging. Animals were anesthetized using isoflurane anesthesia and placed in a PerkinElmer IVIS (in vivo imaging system) Spectrum CT (Waltham, MA). AF750 signal was collected every other day until signal was at or below signal from saline-injected animals or animals reached endpoint due to tumor growth.

Micro-computed tomography imaging. Animals were perfused with D-PBS at the heart followed by neutral buffered formalin (Thermo Fisher Scientific, Inc.) for 5 min, then with saline to rinse, and MicroFil contrast agent (Flow Tech Inc., Carver, MA) catalyzed at a viscosity appropriate for small vessels (5 mL lead-based contrast agent, 2.5 mL diluent, 0.25 mL curing agent). Perfused mice were carefully stored at 4° C. overnight to cure. The following day, tumor samples were excised, and imaged using a SCANCO Medical μCT50 (Scanco USA, Inc., Wayne, PA). μCT image slices were constrained using manual selection of the sample outline and processed with a Gaussian filter at a consistent global threshold via the Scanco Medical μCT Evaluation Program before 3-dimensional reconstruction.

Cytokine analysis. Tumor, LN, and spleen samples were excised from animals, flash frozen using liquid nitrogen, and stored at −80° C. BioPlex lysis buffer (Bio-Rad) was prepared and 100 μL added to each sample. Tissues were mashed using P200 pipette tips until a smooth solution formed. Samples were oscillated at 4° C. for 20 min, spun down, and tissue lysate collected and frozen at −80° C. The following day, total protein content was measured using Pierce bicinchoninic acid assay (Thermo Fisher Scientific, Inc.). Milliplex MAP 32-plex mouse cytokine/chemokine magnetic bead panel (Millipore Sigma) was used to assess cytokine and chemokine content in samples. In brief, samples were added to plate at appropriate dilutions with premixed magnetic beads (Milliplex) in assay buffer (Milliplex) and incubated on a plate shaker overnight at 4° C. Samples were then placed in magnetic plates, decanted, and washed. Detection antibodies (Milliplex) were added to each sample and incubated for 1 h at 4° C. on plate shaker. Streptavidin-phycoerythrin (Milliplex) was added directly to each well, and incubated for 30 min at 4° C. on plate shaker. Samples were then placed in magnetic plate, decanted, and washed. Samples were resuspended in 100 μL drive fluid and analyzed using a MagPix system (Luminex, Austin, TX).

Therapeutic studies. E0771 tumor cells (50,000 cells) were implanted into immunocompetent C57/Bl6 mice in saline, MT, PEG-RGD, and PEG-RDG, and tumors measured every 48 h. Animals were randomized among cages and researchers were blinded to groups. Animals were randomly pre-assigned to a therapeutic group. Once tumors reached ˜100 mm³ in ellipsoidal volume (treatment day 0 [d0]), animals received first treatment of CpG, ICB, or isotype mAb based on pre-assigned therapeutic group. Animals assigned to receive CpG therapy received 3 μg ODN 1826 (CpG, InvivoGen, Inc., San Diego, CA) in 30 μL sterile saline intratumorally on d0 and d7. Animals assigned to receive ICB therapy were given 100 μg each of αPD1 (BioXCell) and αCTLA-4 (clone 9H10, BioXCell) in 100 μL sterile saline intraperitoneally on day 0, 3, and 6. Animals assigned to isotype mAb group were given 100 μg each of rat IgG2a anti-trinitrophenol (BioXCell) and polyclonal Armenian hamster IgG (BioXCell) intraperitoneally on day 0, 3, and 6. Tumor volume and animal weight was monitored every 48 h until animals reached the predetermined endpoint (tumor size of 15 mm in any dimension or if the animal displayed signs of illness or distress).

Statistical analysis. Data are represented as the mean accompanied by SEM, and statistics were calculated using GraphPad Prism 6 and 8 software (GraphPad Software, Inc.). Statistical significance was defined as p<0.05, 0.01, 0.005, and 0.001 based on ANOVA with Tukey's post-hoc test unless otherwise specified.

### Example 2: Improved Consistency and Rate of Formation of Immune Responses in Tumors Implanted in Hydrogels

A preclinical trial was conducted to determine the consistency and reliability of current technologies for the implantation of tumors.

E0771, murine triple-negative breast cancer, cells were implanted into immunocompetent C57/Bl6 and tumor formation rates assessed based on palpation. The rate of tumor formation of E0771 cells implanted in saline or Matrigel is 0-50% or 75-90%, respectively (FIG. 1f). In order to evaluate the consistency of tumor latency for tumors implanted in Matrigel, which demonstrated a higher rate of tumor formation, two batches of Matrigel were procured and tumors implanted in each batch, and growth measured every 48 hours until endpoint (1500 mm in any direction) was reached. This revealed batch-to-batch variations in tumor latency, increasing the potential for these tumors to be unreliable and results not repeatable. E0771 cells were next implanted into immunocompetent C57/Bl6 mice within PEG hydrogels functionalized with RGD (PEG-RGD) or PEG hydrogels functionalized with RDG (PEG-RDG). This revealed 100% tumor formation rates across multiple batches of PEG hydrogel (FIG. 5b). Tumor growth rates were also assessed within these engineered hydrogels and revealed equivalent growth between two batches of PEG hydrogel prepared >1 year apart (FIG. 2d) but differing based on functionalization (FIG. 2d). Thus, not only did the engineered hydrogels induce more consistent tumor formation and growth, but the functionalization within the gels altered tumor growth kinetics.

Flow cytometry was then utilized to assess immune infiltration into scaffolds, which must be consistent for immunotherapeutic responses to be reliable and repeatable. After E0771 tumor implantation in saline, total lymphocyte (CD45+), macrophage, dendritic cell, and T cell infiltration all varied by >1 order of magnitude in scale (FIG. 1j), indicating a high degree of variability, contributing to downfalls in repeatability of current technologies. After implantation of E0771 cells in two different batches of Matrigel, less variability was observed; however, the number of macrophages and T cells within each gel varied significantly (FIG. 1j), likewise indicating high variability and poor reproducibility. In order to evaluate the engineered hydrogel formulations, tumors were implanted in two different batches of PEG hydrogel prepared >1 year apart, and flow cytometry was again utilized to assess immune infiltration into the scaffolds.

This revealed no significant differences in total lymphocyte (CD45+), macrophage, dendritic cell, or T cell infiltration (FIG. 1i), pointing to these scaffolds as inducing less variable and more reliable tumor immune microenvironments.

### Example 3: Functionalization of Engineered Hydrogels Directs Immune Responses

In order to assess the potential for this system to direct immune responses within tumors as they develop, E0771 tumors were implanted within engineered PEG hydrogels and multiple immune analysis techniques utilized to interrogate immune infiltration throughout tumor development and progression.

The phenotype of myeloid cells within tumors has been shown to be important in certain immunotherapy responses, and as such 13-color flow cytometry was utilized to assess the phenotype of dendritic cells, macrophages, and subtype of CD11b+ myeloid cells within tumor microenvironments. This revealed that PEG-RGD scaffolds induced higher dendritic cell activation (FIG. 4a) but equivalent macrophage polarization (FIG. 4b), as assessed by CD206 and CD86 expression. PEG-RDG scaffolds, however, induced greater neutrophil infiltration (FIG. 4c). T cells exert the majority of anti-tumor effects in immunotherapy responses, via direct cytotoxic effects, and as such were assessed within these hydrogels. This revealed enhanced CD8+ T cell infiltration and CD44− CD8+ T cell infiltration within PEG-RGD tumors. Thus, on a cellular basis, PEG-RGD functionalized scaffolds induced a CD86+ dendritic cell and CD8+ T cell response, while PEG-RDG functionalized scaffolds induced a more neutrophil predominant response. Cytokines, which are both produced by and direct immune responses, were next assessed, and revealed more cytokine responses (FIG. 4g), particularly among Th1 cytokines (FIG. 4h), within PEG-RGD scaffolds. Of note, CXCL9, a chemokine produced by neutrophils, was the only cytokine analyzed to show a greater response in PEG-RDG scaffolds (FIG. 4g-h).

This is further indicative of the RDG-functionalized scaffold inducing a neutrophil driven response, with the RGD-functionalized scaffold inducing a predominantly Th1 response.

### Example 4: Use of Engineered Scaffolds Allows for Different Responses Based on Class of Hydrogel and Local Immune Microenvironment

In order to evaluate the influence of engineered tumor immune microenvironments on responses to different classes of immunotherapy, an intratumorally administered CpG vaccine, systemically administered immune checkpoint blockade therapy, and systemically administered isotype monoclonal antibody were applied to tumors implanted in saline, Matrigel, and RGD or RDG-functionalized PEG hydrogel scaffolds.

Application of intratumorally administered CpG as an adjuvant-based cancer vaccine did not result in responses in tumors developed in saline or Matrigel (FIG. 10a). Likewise, responses in RGD-functionalized scaffolds which had a predominantly Th1 immune infiltration, were minimal (FIG. 10a). However, in RDG-functionalized scaffolds, CpG administration resulted in a decrease in tumor volume (FIG. 10a) and provided survival benefits (FIG. 10b), showing that tumors with a predominantly neutrophil-driven response, lacking a Th1 response are more suited for vaccination strategies. A combination of immune checkpoint blocking anti-cytotoxic T lymphocyte antigen-4 (CTLA-4) and anti-programmed death-1 (PD-1) monoclonal antibodies administered systemically did not induce responses in tumors implanted in saline or Matrigel (FIG. 10c). Likewise, anti-CTLA-4+anti-PD-1 did not result in improved responses in tumors in RDG-functionalized tumors (FIG. 10c). However, in RGD-functionalized tumors with predominantly Th1 responses, tumor volume was decreased (FIG. 10c) and survival was enhanced (FIG. 10d), indicating combination anti-CTLA-4 and anti-PD1 monoclonal antibody administration may be an effective strategy for the treatment of tumors with a predominantly Th1 phenotype. Systemic administration of isotype monoclonal antibodies did not result in responses in any tumors assessed (FIG. 10e-f), showing that responses were due to the application of the specific immunotherapies utilized here.

To date, murine tumors are implanted in saline (water with sodium and chloride) or Matrigel (decellularized matrix generated from murine sarcomas). The scaffold harnesses a synthetic hydrogel system with highly controllable degradation rates and adhesive ligands to direct immune infiltration, highly improving upon current variable and uncontrollable systems that are widely used in the field. No other scaffold for tumor implantation allows for control over degradation rate of local scaffolds. Other scaffolds induce highly variable immune infiltration (10+ fold differences in infiltration of immune cell subtypes), while this scaffold induces consistent and controllable immune infiltration, which provides the advantage of immune microenvironments and disease load to be reproducibly induced for in vivo modeling and drug testing. Likewise, this scaffold induces identical tumor growth and latency, while prior techniques induce highly variable tumor growth rates. No other techniques are able to model the tumor microenvironment as consistently or controllably.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLES
Table 1. Statistics between tumors formed within PEG-RGD and PEG-RDG hydrogel
matrix vehicles in FIG. 4g by mixed-effects model with Tukey's post-hoc test.

| | | day 2 | | day 7 | | day 28 | |
|---|---|---|---|---|---|---|---|
| | | p-value | significance | p-value | significance | p-value | significance |
| Inflammatory | IFNg | 0.0287 | * | 0.8809 | | 0.998 | |
| | TNFa | 0.0366 | * | 0.1812 | | 0.996 | |
| | GMCSF | >0.9999 | | >0.9999 | | >0.9999 | |
| | IL1b | >0.9999 | | >0.9999 | | >0.9999 | |
| | IL6 | 0.9999 | | 0.0001 | *** | 0.3863 | |
| | IL9 | >0.9999 | | 0.9971 | | >0.9999 | |
| | IL3 | 0.9996 | | >0.9999 | | >0.9999 | |
| | IL12p40 | 0.9992 | | 0.9905 | | >0.9999 | |
| | IL12p70 | 0.9997 | | 0.9997 | | >0.9999 | |
| Suppressive | IL1a | <0.0001 | **** | >0.9999 | | >0.9999 | |
| | IL2 | 0.9309 | | 0.9719 | | >0.9999 | |
| | IL4 | <0.0001 | **** | 0.459 | | >0.9999 | |
| | IL5 | >0.9999 | | >0.9999 | | 0.6536 | |
| | IL10 | <0.0001 | **** | >0.9999 | | >0.9999 | |
| | IL13 | 0.0102 | * | 0.0016 | *** | >0.9999 | |
| | IL15 | >0.9999 | | 0.9865 | | >0.9999 | |
| Growth | GCSF | >0.9999 | | 0.9692 | | 0.9992 | |
| Factors | LIF | >0.9999 | | 0.9999 | | >0.9999 | |
| | MCSF | 0.7044 | | 0.9919 | | 0.9617 | |
| | VEGF | 0.0002 | *** | 0.9965 | | 0.9996 | |
| Chemokines | IL17 | >0.9999 | | >0.9999 | | >0.9999 | |
| | CCL2 | >0.9999 | | >0.9999 | | >0.9999 | |
| | CCL3 | >0.9999 | | >0.9999 | | >0.9999 | |
| | CCL4 | >0.9999 | | >0.9999 | | 0.999 | |
| | CXCL2 | 0.9694 | | >0.9999 | | >0.9999 | |
| | CXCL5 | 0.9985 | | 0.9973 | | >0.9999 | |
| | CXCL9 | 0.0453 | * | 0.9978 | | 0.0394 | * |
| | CXCL10 | <0.0001 | **** | 0.9653 | | >0.9999 | |
| | Eotaxin | 0.9996 | | >0.9999 | | >0.9999 | |
| | KC | 0.1191 | | 0.0419 | * | >0.9999 | |
| | RANTES | <0.0001 | **** | 0.0056 | ** | 0.2145 | |

* indicates p < 0.05,
** indicates p < 0.01,
*** indicates p < 0.005,
**** indicates p < 0.001,
no notation indicates p > 0.05.

SEQUENCES

```
  1. SEQ ID NO: 1-GRGDSPC
  2. SEQ ID NO: 2-GRDGSPC
  3. SEQ ID NO: 3-GCRDVPMSMRGGDRCG
  4. SEQ ID NO: 4-AGAV
  5. SEQ ID NO: 5-PHSRN
  6. SEQ ID NO: 6-GFOGER
  7. SEQ ID NO: 7-GTPGPQGIAGQRGVV
  8. SEQ ID NO: 8-YIGSR
  9. SEQ ID NO: 9-IKVAV
10. SEQ ID NO: 10-RKRLQVQLSIRT
11. SEQ ID NO: 11-PPFLMLLKGSTR
12. SEQ ID NO: 12-GKKQRFRHRNRKG
13. SEQ ID NO: 13-FHRRIKA
14. SEQ ID NO: 14-GWQPPARARI
15. SEQ ID NO: 15-GCRDGPQGIWGQDRCG
16. SEQ ID NO: 16-GCRDIPESLRAGDRCG
```

1. A cellular matrix scaffold comprising a polyethylene glycol (PEG) hydrogel conjugated to a cell adhesive peptide, wherein the PEG hydrogel further comprises a crosslinking peptide and a population of cancer cells.

2. The cellular matrix scaffold of claim 1, wherein the cell adhesive peptide is selected from the group consisting of a fibronectin-derived peptide, a collagen-derived peptide, a laminin-derived peptide, a glycosaminoglycan binding peptide, and any combination thereof.

3. The cellular matrix scaffold of claim 2, wherein the fibronectin-derived peptide comprises a RGD peptide or a RDG peptide.

4. The cellular matrix scaffold of claim 3, wherein the RGD peptide comprises SEQ ID NO: 1.

5. The cellular matrix scaffold of claim 3, wherein the RDG peptide comprises SEQ ID NO: 2.

6. The cellular matrix scaffold of claim 1, wherein the crosslinking peptide is a bis-cysteine crosslinking peptide.

7. The cellular matrix scaffold of claim 6, wherein the bis-cysteine crosslinking peptide is a VPM peptide.

8. The cellular matrix scaffold of claim 1, wherein the crosslinking peptide comprises SEQ ID NO: 3.

9. The cellular matrix scaffold of claim 1, wherein the PEG is a four-armed maleimide-terminated PEG (PEG-4MAL) macromer.

10. The cellular matrix scaffold of claim 1, wherein the PEG, the cell adhesive peptide, the crosslinking peptide, and the population of cancer cells are combined at a 2:1:1:1 volume ratio.

11. (canceled)

12. A method of generating or inducing a tumor growth in an animal, the method comprising administering a cellular matrix scaffold into the animal, wherein the cellular matrix scaffold comprises a polyethylene glycol (PEG) hydrogel conjugated to a cell adhesive peptide, and wherein the PEG hydrogel further comprises a crosslinking peptide and a population of cancer cells.

13. The method of claim 12, wherein the cell adhesive peptide comprises a RGD peptide or a RDG peptide.

14. The method of claim 12, wherein the RGD peptide comprises SEQ ID NO: 1.

15. The method of claim 12, wherein the RDG peptide comprises SEQ ID NO: 2.

16-21. (canceled)

22. A method of screening a therapeutic agent, the method compromising:
generating a cellular matrix scaffold comprising a polyethylene glycol (PEG) hydrogel conjugated to a cell adhesive peptide, wherein the PEG hydrogel further comprises a crosslinking peptide and a population of cancer cells;
administering the cellular matrix scaffold into an animal to induce a tumor;
monitoring the animal for growth of the tumor; and
administering to the animal a therapeutic agent to treat the tumor.

23. The method of claim 22, wherein the cell adhesive peptide comprises a RGD peptide or a RDG peptide.

24. The method of claim 23, wherein the RGD peptide causes a T cell or a dendritic cell to infiltrate the tumor.

25. The method of claim 23, wherein the RDG peptide causes a neutrophil cell to infiltrate the tumor.

26-29. (canceled)

30. The method of claim 22, wherein the tumor is a breast tumor or a melanoma tumor.

31. The method of claim 22, wherein the method further comprises administering an immunomodulating agent, an adhesive ligand, or a combination thereof.

32-35. (canceled)
